(19) United States
(12) Patent Application Publication
(10) Pub. No.: US 2016/0327572 A1
(43) Pub. Date: Nov. 10, 2016

(54) Biomarkers for Dementia and Dementia Related Neurological Disorders

(71) Applicant: CHILDREN'S MEDICAL CENTER CORPORATION, Boston, MA (US)

(72) Inventors: Omar S. Barnaby, Somerville, MA (US); Judith Steen, Brighton, MA (US); Beth Stevens, Milton, MA (US); Hanno Steen, Brighton, MA (US); Soyon Y. Hong, Brookline, MA (US)

(21) Appl. No.: 15/109,480

(22) PCT Filed: Jan. 6, 2015
(86) PCT No.: PCT/US15/10288

§ 371 Date: Jul. 1, 2016

Related U.S. Application Data
(60) Provisional application No. 61/923,995, filed on Jan. 6, 2014.

(51) Int. Cl. G01N 33/68

(52) U.S. Cl. CPC G01N 33/6896; G01N 2800/2821; G01N 2800/50; G01N 2800/60

(57) ABSTRACT

The present invention provides CSF protein-based biomarkers and biomarker combinations that are useful in diagnosing dementia or a dementia related neurological disorder a patient. In particular, the biomarkers of this invention are useful to classify a subject sample as Alzheimer's dementia, non-Alzheimer's dementia, as Progressive supranuclear palsy (PSP), non-PSP dementia or normal. In some aspects, the invention relates to methods useful for diagnosing, classifying, and profiling dementia or a dementia related neurological disorder a patient.

### CLAIM OF PRIORITY

This application claims the benefit of U.S. Provisional Patent Application Ser. No. 61/923,995, filed on Jan. 6, 2014, the entire contents of which are hereby incorporated by reference.

### TECHNICAL FIELD

This invention relates to protein or peptide molecules (i.e., biomarkers) that are found to have significantly different abundances between dementia or other dementia related neurological disorders, and normal patients. The present invention also relates to methods and kits useful for diagnosing, classifying, and profiling dementia and other dementia related neurological disorders. In some aspects, the invention relates to methods and kits useful for diagnosing, classifying, profiling, and monitoring the progression or regression of between dementia or other dementia related neurological disorders (e.g., Alzheimer's disease (AD), Progressive supranuclear palsy (PSP) and Huntington's Disease (HD)).

### BACKGROUND

Dementia and other cognitive disorders is a serious medical issue that is of increasing concern. Dementia is not a specific disease. It is a descriptive term for a collection of symptoms that can be caused by a number of disorders that affect the brain. People with dementia have significantly impaired intellectual functioning that interferes with normal activities and relationships. They also lose their ability to solve problems and maintain emotional control, and they may experience personality changes and behavioral problems, such as agitation, delusions, and hallucinations.

AD, PSP and HD are diseases that result in dementia. Alzheimer's disease, also referred to as Alzheimer's dementia or AD is a progressive degenerative disease of the brain primarily associated with aging. AD is one of several disorders that cause the gradual loss of brain cells and is a leading cause of dementia. Clinical presentation of AD is characterized by loss of memory, speech, cognition, reasoning, judgment, and orientation. Mild cognitive impairment (MCI) is often the first identified stage of AD. As the disease progresses, motor, sensory, and linguistic abilities also are affected until there is global impairment of multiple cognitive functions. These cognitive losses occur gradually, but typically lead to severe impairment and eventual death in the range of three to twenty years.

HD is a progressive neurodegenerative genetic disorder that affects muscle coordination and leads to cognitive decline and psychiatric problems. Symptoms of the disease can vary between individuals and even among affected members of the same family, but usually progresses predictably. The earliest symptoms are often subtle problems with mood or cognition. A general lack of coordination and an unsteady gait often follows. As the disease advances, uncoordinated, jerky body movements become more apparent, along with a decline in mental abilities and behavioral and psychiatric problems.

PSP is a rare brain disorder that causes serious and progressive problems with control of gait and balance, along with complex eye movement and thinking problems. One of the most obvious sign of the disease is an inability to aim the eyes properly, which occurs because of lesions in the area of the brain that coordinates eye movements. Other symptoms of progressive supranuclear palsy include alterations of mood and behavior (e.g., depression, apathy, cognitive impairments, and/or progressive mild dementia). The prevalence of PSP in the US is 1 per 100,000 in patients over 60 (˜20,000 cases). Pathologically, there is severe neuronal loss in the substantia nigra, globus pallidus, subthalamic nucleus, midbrain, and pontine reticular formation with frequent neurofibrillary tangles composed of straight tau filaments. There is currently no test to definitively diagnose PSP, so physicians focus on ruling out other conditions. An accurate diagnosis of PSP often is not reached until it progresses to the stage when the eyes begin to function poorly. Until then, it is often misdiagnosed as Parkinson's.

Biomarkers that can be used to diagnose, or monitor the progression or regression of dementia (e.g., age-related dementia) and/or other dementia related neurological disorders are in demand. An early diagnosis of dementia has many advantages including, for example, increased time to maximize quality of life, reduced anxiety about unknown problems, increased chances of benefiting from treatment and increased time to plan for the future. However, reliable and noninvasive methods for diagnosing dementia are not available. Thus, a need exists for methods which diagnose dementia, (e.g., AD, HD, PSP) before significant neuronal loss has occurred.

### SUMMARY

The present invention is based, at least in part, on the discovery that proteins present in CSF, including those of the classical and alternative complement pathway, are biomarkers useful for identifying a subject's risk of developing dementia (e.g., age related dementia, Alzheimer's disease (AD), Progressive supranuclear palsy (PSP), or Huntington's Disease (HD)).

In one aspect, the disclosure provides a method for determining the risk of developing dementia or other dementia related neurological disorders in a subject, the method comprising obtaining a sample from the subject, determining a level of one or more biomarkers in said sample, comparing the levels of the one or more biomarkers with reference levels of the same biomarkers to identify an increase or decrease in a level of said one or more biomarkers is said sample; and identifying a subject who has an increase or decrease in the level of said one or more biomarkers is said sample as having an increased risk of developing dementia. The dementia or a dementia related neurological disorder contemplated herein include, but are not limited to, AD, PSP, HD, dementia of mixed type, Parkinson's Disease, diffuse Lewy Body dementia, vascular dementia, frontotemporal dementia, semantic dementia and dementia with Lewy bodies. In exemplary embodiments, the methods disclosed herein relate to determining the risk of developing AD, HD or PSP. The methods disclosed herein can also be used to monitor the progression or regression dementia or other dementia related neurological disorders in a subject.

In one aspect, the methods provided herein comprise determining the level of one or more biomarkers selected from the group of biomarkers listed in Tables 1-4. In some embodiments, the methods comprise determining the levels of one or more of Neuroserpin (NEUS), Guanine deaminase (GUAD), N(G),N(G)-dimethylarginine dimethylaminohydrolase 1 (DDAH1), V-type proton ATPase subunit 1 (VAS1), Complement C1q tumor necrosis factor-related protein 3 (C1QT3), hemoglobin subunit delta (HBD), hemoglobin subunit alpha (HBA), Transmembrane protein 132A (T132A), Keratin, type I cytoskeletal 17 (K1C17), Ig lambda chain V-III region SH (LV301), Keratin, type I cytoskeletal 16 (K1C16), Inter-alpha-trypsin inhibitor heavy chain H4 (ITIH4), Immunoglobulin lambda-like polypeptide 5 (IGLL5), Ig kappa chain V-III region VG (Fragment) (KV309), Collagen alpha-2(VI) chain (CO6A2), Ig alpha-1 chain C region (IGHA1), and Ig alpha-1 chain C region (IGHA1).

An increase in the level of one or more of NEUS, GUAD, DDAH1, VAS1, or Complement C1q tumor necrosis factor-related protein 3 (C1QT3) indicates that the subject has an increased risk of developing Alzheimer's Disease (AD).

A decrease in the level of level of one or more of HBD, HBA, T132A, K1C17, LV301, K1C16, ITIH4, IGLL5, KV309, CO6A2, IGHA1, or IGLL5 indicates that the subject has an increased risk of developing Alzheimer's Disease (AD).

In some other embodiments, the methods comprise determining the levels of one or more of Phosphoinositide-3-kinase-interacting protein 1 (P3IP1), Secretogranin-1 (SCG1), Serine/threonine-protein kinase LATS2 (LATS2), Neogenin (NEO1), UPF0764 protein C16orf89 (CP089), Keratin, type I cytoskeletal 17 (K1C17), Golgi membrane protein 1 (GOLM1), L-lactate dehydrogenase A chain (LDHA), Disintegrin and metalloproteinase domain-containing protein 22 (ADA22), GDNF family receptor alpha-2 (GFRA2), Neurosecretory protein VGF (VGF), Superoxide dismutase [Mn], mitochondrial (SODM), Neuronal growth regulator 1 (NEGR1), Amyloid beta A4 protein (A4), Cell adhesion molecule 1 (CADM1), Transcriptional activator GLI3 (GLI3), Neuronal pentraxin-1 (NPTX1), Neural cell adhesion molecule L1-like protein (CHL1), Chondroitin sulfate proteoglycan 5 (CSPG5), Receptor-type tyrosine-protein phosphatase gamma (PTPRG), Calsyntenin-1 (CSTN1), Tyrosine-protein phosphatase non-receptor type substrate 1 (SHPS1), Cathepsin F (CATF), Tenascin-X (TENX), Protein FAM3C (FAM3C), Multiple epidermal growth factor-like domains protein 8 (MEGF8), Neuronal cell adhesion molecule (NRCAM), Neuronal pentraxin receptor (NPTXR), Neuropilin-1 (NRP1), Uncharacterized protein C14orf37 (CN037), Protein kinase C-binding protein NELL2 (NELL2), Alpha-1-antitrypsin (A1AT), Thyroxine-binding globulin (THBG), Ig mu chain C region (IGHM), Ig kappa chain V-III region VG (Fragment) (KV309), Collagen alpha-2(VI) chain (CO6A2), Ig alpha-1 chain C region (IGHA1), Ig heavy chain V-III region TIL (HV304), hemoglobin subunit beta (HBB), and Haptoglobin (HPT).

A decrease in the level of one or more of A4, ADA22, C1RL, CADM1, CANT1, CATF, CHL1, CN037, CP089, CSPG5, CSTN1, F13A, FAM3C, GFRA2, GLI3, GOLM1, HBD, IGHA1, IGHA2, K1C17, LATS2, LDHA, LYVE1, MEGF8, NEC1, NEGR1, NELL2, NEO1, NPTX1, NPTXR, NRCAM, NRP1, P3IP1, PTPRG, PTPRN, SCG1, SCG2, SHPS1, SMS, SODM, TENX, TICN2, VGF and VWF indicates that the subject has an increased risk of developing PSP.

An increase in the level of one or more of A1AT, THBG, IGHM, KV309, HBB, HV304 or HPT indicates that the subject has an increased risk of developing PSP.

In one aspect, the methods provided herein comprise determining the level of one or more complement factors selected from the group consisting of Factor H (related protein 2), C3a anaphylatoxin, C8 alpha chain, C8 beta chain, Factor B, CD59, C4a, C1q tumor necrosis factor, C6, C8 and C9. An increase in any one of Factor H (related protein 2), C8 alpha chain, C8 beta chain, C3a anaphylatoxin Factor B, C4a, C6, C8 or C9 indicates that the subject has an increased risk of developing PSP. An increase in any one of CD59 or C1q tumor necrosis factor (C1QT3) indicates that the subject has an increased risk of developing Alzheimer's disease (AD).

In some aspects, the methods further comprise selecting a treatment for the subject based on the comparison of the levels of the biomarkers with the reference levels. For subjects identified as having an increased risk of developing AD, PSP or HD, the methods may further comprise selecting a treatment plan for a subject. For example, the methods may further comprise selecting a treatment plan for a subject, wherein the treatment plan comprises selectively administering a composition comprising an effective amount of a therapeutic agent. In some embodiments, the treatment plan can comprise administering the selected treatment to the subject (i.e., administering an anti-dementia compound selected from small molecules (e.g., donepezil, memantine, rivastigmine, galanthamine, tacrine, or salts thereof), inhibitory nucleic acids, antibodies, and inhibitory peptides) In some embodiments, treatment plan can comprise administering the Thus, the methods presented herein may further comprise selectively administering a composition comprising an effective amount of an anti-dementia compound selected from the group consisting of donepezil, memantine, rivastigmine, galanthamine, and tacrine, or salts thereof to a subject identified as having an increased risk of developing AD, PSP or HD.

In some embodiments, the reference level of the one or more biomarkers is determined from a sample obtained from a non-demented control subject.

The term “subject” as used herein refers to a mammal A subject therefore refers to, for example, dogs, cats, horses, cows, pigs, guinea pigs, and the like. The subject can be a human. When the subject is a human, the subject may be referred to herein as a patient. The subject can be symptomatic (e.g., the subject presents symptoms associated with dementia or dementia related neurological disorders), or the subject can be asymptomatic (e.g., the subject does not present symptoms associated with dementia or dementia related neurological disorders).

As used herein, the term “biological sample” or “sample” refers to a sample obtained or derived from a patient. By way of example, the sample may be selected from the group consisting of body fluids, cerebrospinal fluid sample (CSF), blood, whole blood, plasma, serum, mucus secretions or saliva. In some embodiments the sample is, or comprises a CSF sample.

The terms “biomarker”, “marker” or “biochemical marker” refers to any enzyme, protein, polypeptide, peptide, isomeric form thereof, immunologically detectable fragments thereof, or other molecule, whose presence, absence, or variance in body fluids from so-called “reference” (i.e., “normal”) levels, are indicative of dementia or dementia related neurological disorder.

In some embodiments, the reference sample is obtained from at least one individual not suffering from dementia or dementia related neurological disorders. In some other embodiments, the reference sample is obtained from at least one individual previously diagnosed as having dementia or a dementia related neurological disorders. In some embodiments, the reference sample comprises a predetermined, statistically significant reference analyte levels.

The levels of the biomarkers for a subject can be obtained by any art recognized method. The level can be determined by measuring the level of the biomarker(s) in a body fluid (clinical sample), e.g., CSF or serum. The level of biomarkers can be determined (e.g., measured) by any suitable methods and materials known in the art, including, for example, a process selected from the group consisting of mass spectrometry, ELISA, immunoassays, enzymatic assays, spectrophotometry, colorimetry, fluorometry, bacterial assays, compound separation techniques, or other known techniques for determining the presence and/or quantity of an analyte.

In some embodiments, the levels are measured using mass spectrometry (MS) analysis. The MS analysis method can be liquid chromatography-tandem mass spectrometry (UPLC-MS/MS) or gas chromatography-mass spectrometry (GC-MS).

The preferred biological source for detection of the biomarkers is cerebrospinal fluid (“CSF”). However, in other embodiments, the biomarkers can be detected in serum. Many of the biomarkers of the present invention can be found in both CSF and serum.

In some embodiments, the methods provided herein further comprise selecting a treatment for the subject based on the comparison of the levels of the biomarkers with the reference levels, followed by administering the selected treatment to the subject. In one embodiment, the methods comprise administering to the subject an effective amount of at least one anti-dementia compound.

In another aspect, the invention provides kits for evaluating a human subject for being at risk of developing AD, PSP or HD. The kits include reagents suitable for determining levels of a plurality of analytes in a test sample (.e.g., reagents suitable for determining levels of the biomarkers disclosed herein); optionally one or more control samples comprising predetermined levels of the same analytes, wherein comparison of the levels of the analytes in a test sample with levels in the control samples identifies a subject as being at risk of developing AD, PSP or HD; and instructions for use of the kit in the method described herein.

The section headings used herein are for organizational purposes only and are not to be construed as limiting the described subject matter in any way. When definitions of terms in incorporated references appear to differ from the definitions provided in the present teachings, the definition provided in the present teachings shall control. It will be appreciated that there is an implied “about” prior to metrics such as temperatures, concentrations, and times discussed in the present teachings, such that slight and insubstantial deviations are within the scope of the present teachings herein. In this application, the use of the singular includes the plural unless specifically stated otherwise. Also, the use of “comprise,” “comprises,” “comprising,” “contain,” “contains,” “containing,” “include,” “includes,” and “including” are not intended to be limiting. It is to be understood that both the foregoing general description and the following detailed description are exemplary and explanatory only and are not restrictive of the invention. The articles “a” and “an” are used herein to refer to one or to more than one (i.e., to at least one) of the grammatical object of the article. By way of example, “an element” means one element or more than one element.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

The patent or application file contains at least one drawing executed in color. Copies of this patent or patent application publication with color drawing(s) will be provided by the Office upon request and payment of the necessary fee.

FIG. 1 is a graph demonstrating proteins showing the largest up-regulation in PSP. (CO4A=Complement C4-A; SPRC=SPARC; K2C1=Keratin, type II cytoskeletal 1; A1AG1=Alpha-1-acid glycoprotein 1; A1AT=Alpha-1-antitrypsin; THBG=Thyroxine-binding globulin; IGHM=Ig mu chain C region; KV309=Ig kappa chain V-III region VG (Fragment); HV304=Ig heavy chain V-III region TIL; and HPT=Haptoglobin).

FIG. 2 is a graph demonstrating proteins showing the largest down-regulation in PSP. (P3IP1=Phosphoinositide-3-kinase-interacting protein 1; SCG1=Secretogranin-1; LATS2=Serine/threonine-protein kinase LATS2; NEO1=Neogenin; CP089=UPF0764 protein C16orf89; K1C17=Keratin, type I cytoskeletal 17; GOLM1=Golgi membrane protein 1; LDHA=L-lactate dehydrogenase A chain; and ADA22=Disintegrin and metalloproteinase domain-containing protein 22).

FIG. 3 is a graph demonstrating proteins showing the largest up-regulation in Alzheimer's disease. (RARR2=Retinoic acid receptor responder protein 2; CSF1R=Macrophage colony-stimulating factor 1 receptor; ENOG=Gamma-enolase; CYTM=Cystatin-M; PGSQ=Decorin; NEUS=Neuroserpin; GUAD=Guanine deaminase; DDAH1=N(G),N(G)-dimethylarginine dimethylaminohydrolase 1; VAS1=V-type proton ATPase subunit 1; and C1QT3=Complement C1q tumor necrosis factor-related protein 3).

FIG. 4 is a graph demonstrating proteins showing the largest down-regulation in Alzheimer's disease. (HBD=hemoglobin subunit delta; HBA=hemoglobin subunit alpha; T132A=Transmembrane protein 132A; K1C17=Keratin, type I cytoskeletal 17; LV301=Ig lambda chain V-III region SH; K1C16=Keratin, type I cytoskeletal 16; ITIH4=Inter-alpha-trypsin inhibitor heavy chain H4; IGLL5=Immunoglobulin lambda-like polypeptide 5; and KV309=Ig kappa chain V-III region VG (Fragment)).

FIG. 5 is a graph demonstrating proteins showing opposing regulation in PSP and AD. (KV309=Ig kappa chain V-III region VG (Fragment); A4=Amyloid beta A4 protein; SHPS1=Tyrosine-protein phosphatase non-receptor type substrate 1; CADM1=Cell adhesion molecule 1; CSTN1=Calsyntenin-1; A1AT=Alpha-1-antitrypsin; and SCG3=Secretogranin-3).

FIG. 6 is an array tomography demonstrating that C1q localizes to synapses in the human AD brain. Colocalization of complement C1q (green) with many pre- and postsynaptic proteins synapsin (white) and PSD95 (red) on a frontal cortex of postmortem AD brain section.

FIG. 7 is a graph demonstrating C1q is specifically upregulated in areas vulnerable to Aβ deposition in young (P30) pre-plaque J20 APP tg mice. Pre-plaque P30 J20 tg and littermate controls show that there is an early and region-specific increased levels of C1q in the hippocampus and prefrontal cortex. DAPI (blue). DG=dentate gyrus, PFC=pre-frontal cortex, STR=striatum, CRB=cerebellum (***P<0.0001 by 2-way ANOVA for genotypre and region, *** P<0.001 by Bonferroni posttest).

FIG. 8 is a series of high-resolution confocal images demonstrating C1q is specifically upregulated in areas vulnerable to Aβ deposition in young (P30) pre-plaque J20 APP tg mice. Pre-plaque P30 J20 tg and littermate controls show that there is an early and region-specific increased levels of C1q in the hippocampus and prefrontal cortex. C1q (green), DAPI (blue). DG=dentate gyrus, PFC=pre-frontal cortex, STR=striatum, CRB=cerebellum.

FIG. 9 (A and B) provides a series of high-resolution confocal images demonstrating that C1q is localized to PSD95 in hippocampus of young (P30) pre-plaque J20 APP tg mice. High-resolution confocal shows that C1q localizes to PSD95 in J20 tg mice at P30 (A). C1q (green), PSD95 (red), DAPI (blue). Quantification of co-localization of C1q with PSD-95 using ImageJ shows that % PSD-95 that is colocalized with PSD-95 is significantly higher in the J20 tg vs. controls (N=4 pairs); *P<0.05 by two-tailed t-test (B).

FIGS. 10A-D are a series of graphs demonstrating region-specific deposition of C1q onto PSD95 in the brains of young (P30) pre-plaque J20 mice. Quantification of co-localization of C1q with PSD-95 using ImageJ shows that % PSD-95 that is colocalized with PSD-95 is significantly higher in the dentate (A) and frontal cortex (B), but not in the striatum (C) or cerebellum (D), of J20 mice vs. littermate WT controls (N=4 per genotype); *P<0.05 by two-tailed t-test.

FIG. 11 is a schematic diagram demonstrating the classical complement cascade.

FIG. 12 provides a series of images and graphs demonstrating C3 and C1q protein levels are elevated in vulnerable regions of BACHD brains. Fluoresence pictographs showing C1q (left panels) and C3 (right panels) staining in the striatum, motor cortex and dentate gyrus (DG) of 13mth BACHD mice and WT littermate controls. C1q and C3 are significantly upregulated in the striatum and motor cortex (vulnerable regions in HD pathology) but not in the DG (less vulnerable in HD). Bar charts showing quantification of C1q (left bar graph) and C3 (right bar graph) puncta in the different regions, showing significantly more C1q and C3 in the striatum and motor cortex of the BACHD mouse than in WT litter mate controls (p<0.05) n=3, Scale bar=20 um.

FIGS. 13A-F provide a series of images and graphs demonstrating colocalisation (“tagging”) of complement cascade components with presynaptic markers V-Glut 1/2 in the striatum of 7 m BACHD mice. Panels A-C show increased localization of both C1q (A and B) and C3 (C and D) to presynaptic terminals in the BACHD striata relative to WT littermate striata. Bar charts showing percentage of total V-Glut 1/2 that is “tagged” with C1q (E) or C3 (F) in the striata of 7 m BACHD and WT mice.

FIGS. 14A-F are a series of bar graphs demonstrating colocalisation of complement cascade components in HD-vulnerable brain regions of zQ175 knock-in mice. (A-B) C3 and C1q protein levels are significantly elevated the striatum, motor cortex and dentate gyrus (DG) of 7 mth zQ175 mice but not WT controls. (C). Bar chart showing increased colocalisation (“tagging”) of C3 with presynaptic markers V-Glut 1/2 in the striata of zQ175 mice. (D-F). Evidence for microglia activation based on increased expression of CD68 in zQ175 mice (E) compared to WT controls (D), and by morphological criteria of microglia activation (F).

### DETAILED DESCRIPTION

The present inventors have developed a novel platform for the analysis of CSF using quantitative mass spectrometry technique using tandem mass tags (TMT) to quantify CSF proteins in an unbiased manner. This platform was used to compare the concentration of proteins in CSF samples obtained from control patients, Alzheimer's patients, and Progressive Supranuclear Palsy patients. As demonstrated herein, the inventors identified 117 proteins that were differentially regulated in PSP CSF and 46 proteins that were differentially regulated in Alzheimer's CSF with a fold change that is greater that 1.2-fold versus control CSF. Of these differentially regulated proteins, 110 were uniquely regulated by 1.2 fold in PSP CSF and 39 were uniquely regulated by 1.2 fold in Alzheimer's CSF. When the fold change threshold is increased to 1.5, 13 proteins were uniquely regulated in AD and 36 proteins were found to be differentially regulated in PSP. Of these proteins, only a single protein, keratin, type 1 cytoskeletal 17, was found to be differentially regulated in both datasets. Several of these proteins are involved in the immune response and complement pathway.

Further, the inventors demonstrate early upregulation of complement components C1q and C3 and activation of the classical complement cascade in transgenic mouse models of AD and HD. Specifically, the inventors show early and region specific upregulation of complement components C1q and C3 in areas vulnerable to Aβ deposition in young (P30) pre-plaque J20 APP mice compared to WT littermate controls. Similarly, the inventors show early and region specific upregulation of complement components C1q and C3 in vulnerable synapses in fl-mHTT HD mouse models (BACHD and zQ175). These data identify novel protein biomarkers of dementia risk. These biomarkers can better help the clinician diagnose, stratify, or monitor the progression or regression of dementia and other dementia related neurological disorders, than currently available assays.

A biomarker is an organic biomolecule which is differentially present in a sample taken from a subject having a disease as compared with a subject not having the disease. A biomarker is differentially present if the mean or median expression level of the biomarker in the different groups is calculated to be statistically significant. Common tests for statistical significance include, among others, t-test (e.g., student t-test), ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for disease (diagnostics), therapeutic effectiveness of a drug and drug toxicity.

The biomarkers of this invention are biomolecules. Accordingly, this invention provides these biomolecules in isolated form. The biomarkers can be isolated from biological fluids, such as CSF or serum. They can be isolated by any method known in the art, based on both their mass and their binding characteristics. For example, a sample comprising the biomolecules can be subject to chromatographic fractionation, as described herein, and subject to further separation by, e.g., acrylamide gel electrophoresis. Knowledge of the identity of the biomarker also allows their isolation by immunoaffinity chromatography.

### Dementia

The invention, in some aspects, relates to methods, compositions and kits useful for diagnosing and/or determining risk of developing dementia or a dementia related neurological disorder. Dementia is not a single disease, but a non-specific syndrome (i.e., set of signs and symptoms). As used herein, “dementia” refers broadly to any disorder, disease, or syndrome characterized by an abnormal high and progressive loss of functional capacity of the brain. While symptoms of dementia can vary greatly, hallmarks of dementia include impairment of several core mental functions, including memory, communication and language, ability to focus and pay attention, reasoning and judgment, and visual perception. People with dementia may have problems with short-term memory, keeping track of a purse or wallet, paying bills, planning and preparing meals, remembering appointments or traveling out of the neighborhood. Many dementias are progressive, meaning symptoms start out slowly and gradually get worse. Dementia may be determined using standard clinical procedures, with the degree of dementia being defined by the score in the Mini Mental State Examine (MMSE), as detailed in Folstein M. F., Folstein S. E. and McHugh P. R., J Psychiatry Res., 12:189-198 (1975). For example, a score of 30 to 27 points in the MMSE is classified as non-demented, a score of 26 to 20 is considered mildly demented, a score of 19 to 10 points is considered moderately demented and a score of 9 to 0 points is considered severely demented. Dementia, as used herein, includes all ranges of scores of the MMSE, except, of course, those scores classified as non-demented. However, “dementia,” as used herein, is not to be limited by the presence or absence of an MMSE score. Other examples include the abbreviated mental test score (AMTS), the, Modified Mini-Mental State Examination (3MS), the Cognitive Abilities Screening Instrument (CAST), the Trail-making test, and the clock drawing test. As used herein, a dementia-related neurological disorder is disease characterized by the presence of dementia.

Examples of dementia or a dementia related neurological disorder include, but are not limited to, Alzheimer's Disease (AD), progressive supranuclear palsy (PSP), Huntington's Disease (HD), dementia of mixed type, Parkinson's Disease, diffuse Lewy Body dementia, vascular dementia, frontotemporal dementia, semantic dementia and dementia with Lewy bodies.

### Detection of Biomarkers

The biomarkers of the invention can be used in diagnostic tests to assess Alzheimer's disease status in a subject, e.g., to diagnose Alzheimer's disease. The phrase “Alzheimer's disease status” includes distinguishing, inter alia, Alzheimer's disease v. non-Alzheimer's disease and, in particular, Alzheimer's disease v. non-Alzheimer's disease normal or Alzheimer's disease v. non-Alzheimer's disease dementia. Based on this status, further procedures may be indicated, including additional diagnostic tests or therapeutic procedures or regimens.

The biomarkers of the invention can be used in diagnostic tests to assess PSP disease status in a subject, e.g., to diagnose PSP. The phrase “PSP disease status” includes distinguishing, inter alia, PSP disease v. non-PSP disease and, in particular, PSP disease v. non-PSP disease normal or PSP disease v. Parkinson's disease dementia. Based on this status, further procedures may be indicated, including additional diagnostic tests or therapeutic procedures or regimens.

The biomarkers of the invention can be used in diagnostic tests to assess the HD status in a subject, e.g., to diagnose HD. The phrase “HD status” includes distinguishing, inter alia, HD v. non-HD patients. Based on this status, further procedures may be indicated, including additional diagnostic tests or therapeutic procedures or regimens.

The level of the one or more biomarkers disclosed herein can be obtained by any art recognized method. In some embodiments, the levels of the biomarkers may be determined, measured, detected and/or quantified by any suitable methods and materials known in the art, including, for example, a process selected from the group consisting of mass spectrometry, ELISA, immunoassays, enzymatic assays, spectrophotometry, colorimetry, fluorometry, compound separation techniques, protein microarrays, or other known techniques for determining the presence and/or quantity of an analyte.

Compound separation techniques yield a time resolved separation of the analytes comprised by the sample. Suitable techniques for separation to be used include, for example, all chromatographic separation techniques such as liquid chromatography (LC), high performance liquid chromatography (HPLC), gas chromatography (GC), thin layer chromatography, size exclusion or affinity chromatography. These techniques are well known in the art and can be applied by the person skilled in the art. In some embodiments, the methods utilize LC and/or GC chromatographic techniques including, for example, gas chromatography mass spectrometry (GC-MS), liquid chromatography mass spectrometry (LC-MS), liquid chromatography-tandem mass spectrometry (UPLC-MS/MS), direct infusion mass spectrometry or Fourier transform ion-cyclotrone-resonance mass spectrometry (FT-ICR-MS), capillary electrophoresis mass spectrometry (CE-MS), high-performance liquid chromatography coupled mass spectrometry (HPLC-MS), quadrupole mass spectrometry, any sequentially coupled mass spectrometry, such as MS-MS or MS-MS-MS, inductively coupled plasma mass spectrometry (ICP-MS), pyrolysis mass spectrometry (Py-MS), ion mobility mass spectrometry or time of flight mass spectrometry (TOF). In some embodiments, LC-MS and/or GC-MS. As an alternative or in addition to mass spectrometry techniques, the following techniques may be used for compound determination: nuclear magnetic resonance (NMR), magnetic resonance imaging (MRI), Fourier transform infrared analysis (FT-IR), ultra violet (UV) spectroscopy, refraction index (RI), fluorescent detection, radiochemical detection, electrochemical detection, light scattering (LS), dispersive Raman spectroscopy or flame ionization detection (FID). These techniques are well known to the person skilled in the art and can be applied without further ado. In some embodiments, the methods disclosed herein shall be, optionally, assisted by automation. For example sample processing or pre-treatment can be automated by robotics. Data processing and comparison can be assisted by suitable computer programs and databases. Automation as described herein allows using the method of the present invention in high-throughput approaches.

Conventional “determining” methods include sending a clinical sample(s) to a commercial laboratory for measurement or the use of commercially available assay kits. Exemplary kits and suppliers will be apparent to the skilled artisan.

In various embodiments, biomarkers may be determined, detected and/or quantified using lateral flow devices, such as for point-of-care use, as well as spot check colorimetric tests.

The methods of the current invention involve obtaining a sample from a patient. The preferred biological source for detection of the biomarkers is cerebrospinal fluid (“CSF”). The sample should comprise cerebrospinal fluid (CSF). Thus, the sample may include additional components or additives that are naturally occurring or are synthetic, or the sample may be pure CSF. The CSF may be processed after it is obtained. Such examples of processing include, but are not limited to, concentrating, diluting, purifying, or admixing the obtained CSF. In other embodiments, the biomarkers can be detected in serum. Many of the biomarkers of the present invention can be found in both CSF and serum.

As used herein, “obtain” or “obtaining” can be any means whereby one comes into possession of the sample by “direct” or “indirect” means. Directly obtaining a sample means performing a process (e.g., performing a physical method such as extraction) to obtain the sample. Indirectly obtaining a sample refers to receiving the sample from another party or source (e.g., a third party laboratory that directly acquired the sample). Directly obtaining a sample includes performing a process that includes a physical change in a physical substance, e.g., a starting material, such as a blood, e.g., blood that was previously isolated from a patient. Thus, obtain is used mean collection and/or removal of the sample from the subject. Examples of obtaining a sample from a subject are readily apparent and include, but are not limited to lumbar puncture procedures (spinal tap). Furthermore, “obtain” is also used to mean where one receives the sample from another who was in possession of the sample previously.

In some cases, the methods disclosed herein involve comparing levels or occurrences (e.g., presence or absence) to a reference. The reference can take on a variety of forms. In some cases, the reference comprises predetermined values for a protein biomarker in a sample. The predetermined value can take a variety of forms. It can be a level or occurrence of the one or more protein biomarkers disclosed herein in a control subject (e.g., a subject with dementia or a dementia related neurological disorder (i.e., an affected subject) or a subject without such a disorder (i.e., a normal subject (i.e., a subject classified as non-demented)). It can be a level or occurrence in the same subject, e.g., at a different time point. A predetermined level can be single cut-off value, such as a median or mean. It can be a range of cut-off (or threshold) values, such as a confidence interval. It can be established based upon comparative groups, such as where the risk in one defined group is a fold higher, or lower, (e.g., approximately 2-fold, 4-fold, 8-fold, 16-fold or more) than the risk in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk. Moreover, the reference could be a calculated reference, most preferably the average or median, for the relative or absolute amount of a biomarker of a population of individuals comprising the subject to be investigated. The absolute or relative amounts of the analytes of said individuals of the population can be determined as specified elsewhere herein. How to calculate a suitable reference value, preferably, the average or median, is well known in the art. The population of subjects referred to before shall comprise a plurality of subjects, preferably, at least 5, 10, 50, 100, 1,000 subjects. It is to be understood that the subject to be diagnosed by the method of the present invention and the subjects of the said plurality of subjects are of the same species.

Subjects associated with predetermined values are typically referred to as control subjects (or controls). A control subject may or may not have a dementia or a dementia related neurological disorder (e.g., AD, HD or PSP). In some cases it may be desirable that control subject has been diagnosed as having dementia or a dementia related neurological disorder, and in other cases it may be desirable that a control subject has not been diagnosed as having dementia or a dementia related neurological disorder.

Thus, in some cases the level of the protein biomarker in a subject being greater than or equal to the level of the protein biomarker in a control subject is indicative of a clinical status (e.g., indicative of a dementia or a dementia related neurological disorder diagnosis). In other cases the level of the protein biomarker in a subject being less than or equal to the level of protein biomarker in a control subject is indicative of a clinical status. The amount of the greater than and the amount of the less than is usually of a sufficient magnitude to, for example, facilitate distinguishing a subject from a control subject using the disclosed methods. Typically, the greater than, or the less than, that is sufficient to distinguish a subject from a control subject is a statistically significant greater than, or a statistically significant less than. In cases where the level of the protein biomarker in a subject being equal to the level of the protein biomarker in a control subject is indicative of a clinical status, the “being equal” refers to being approximately equal (e.g., not statistically different).

The predetermined value can depend upon a particular population of subjects (e.g., human subjects) selected. For example, an apparently healthy population will have a different ‘normal’ range of the protein biomarker than will a population of subjects which have, or are likely to have, a dementia or a dementia related neurological disorder. Accordingly, the predetermined values selected may take into account the category (e.g., healthy, at risk, diseased) in which a subject (e.g., human subject) falls. Appropriate ranges and categories can be selected with no more than routine experimentation by those of ordinary skill in the art.

In some cases a predetermined value of a protein biomarker is a value that is the average for a population of healthy subjects (human subjects) (e.g., human subjects who have no apparent signs and symptoms of dementia or a dementia related neurological disorder). The predetermined value will depend, of course, on the particular protein (biomarker) selected and even upon the characteristics of the population in which the subject lies. In characterizing likelihood, or risk, numerous predetermined values can be established.

A level, in some embodiments, may itself be a relative level that reflects a comparison of levels between two states. Relative levels that reflect a comparison (e.g., ratio, difference, logarithmic difference, percentage change, etc.) between two states (e.g., healthy and diseased) may be referred to as delta values. The use of relative levels is beneficial in some cases because, to an extent, they exclude measurement related variations (e.g., laboratory personnel, laboratories, measurements devices, reagent lots/preparations, assay kits, etc.). However, the invention is not so limited.

Biomarker levels and/or reference levels may be stored in a suitable data storage medium (e.g., a database) and are, thus, also available for future diagnoses. This also allows efficiently diagnosing prevalence for a disease because suitable reference results can be identified in the database once it has been confirmed (in the future) that the subject from which the corresponding reference sample was obtained did have dementia or a dementia related neurologic disorder. As used herein a “database” comprises data collected (e.g., analyte and/or reference level information and/or patient information) on a suitable storage medium. Moreover, the database, may further comprise a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative of dementia or a dementia related neurologic disorder (e.g. a query search). Thus, if an identical or similar data set can be identified in the data collection, the test data set will be associated with dementia or a dementia related neurologic disorder. Consequently, the information obtained from the data collection can be used to diagnose dementia or a dementia related neurologic disorder or based on a test data set obtained from a subject. More preferably, the data collection comprises characteristic values of all analytes comprised by any one of the groups recited above.

The terms “decrease”, “decreased” “reduced”, “reduction” or ‘down-regulated” are all used herein generally to mean a decrease by a statistically significant amount. However, for avoidance of doubt, ““reduced”, “reduction”, “decreased” or “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level, or at least about a 0.5-fold, or at least about a 1.0-fold, or at least about a 1.2-fold, or at least about a 1.5-fold, or at least about a 2-fold, or at least about a 3-fold, or at least about a 4-fold, or at least about a 5-fold or at least about a 10-fold decrease, or any decrease between 1.0-fold and 10-fold or greater as compared to a reference level.

The terms “increased”, “increase” or “up-regulated” are all used herein to generally mean an increase by a statically significant amount; for the avoidance of any doubt, the terms “increased” or “increase” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 0.5-fold, or at least about a 1.0-fold, or at least about a 1.2-fold, or at least about a 1.5-fold, or at least about a 2-fold, or at least about a 3-fold, or at least about a 4-fold, or at least about a 5-fold or at least about a 10-fold increase, or any increase between 1.0-fold and 10-fold or greater as compared to a reference level.

### Diagnosis/Characterization

The present invention relates to methods useful for the characterization (e.g., clinical evaluation, diagnosis, classification, prediction, profiling) of dementia or dementia related neurological disorders (e.g., AD, HD or PSP), based on the levels, presence, or absence of certain protein biomarkers (i.e., one or more biomarkers listed in Tables 1-4) in CSF. As used herein, levels refer to the amount or concentration of a protein biomarkers in a sample (e.g., a CSF sample) or subject. The level may be expressed as an exact quantity, or may be expressed as a ratio to a reference sample. In some cases, the methods can include determining whether protein biomarker is present in a concentration or a ratio above or below a reference level or ratio.

In some embodiments, the methods involve determining the ratio or levels of one or a plurality of biomarkers (e.g., one or more biomarkers listed in Tables 1-4) in a clinical sample, comparing the result to a reference ratio or level, and characterizing (e.g., diagnosing, classifying) the sample based on the results of the comparison. A clinical sample can be any biological specimen (e.g., a CSF sample) useful for characterizing the dementia or dementia related neurological disorder (e.g., AD, HD or PSP). Exemplary biological specimens can include cerebrospinal fluid sample (CSF), blood, whole blood, plasma, serum, mucus secretions or saliva.

In some embodiments, the methods involve diagnosing dementia or dementia related neurological disorder in a subject. To practice the diagnostic methods the levels of a plurality of biomarkers are typically determined. These levels are compared to a reference wherein the levels of the plurality of biomarkers in comparison to the reference is indicative of whether or not the subject has a dementia or dementia related neurological disorder and/or should be diagnosed with dementia or dementia related neurological disorder.

As used herein, diagnosing includes both diagnosing and aiding in diagnosing. Thus, other diagnostic criteria may be evaluated in conjunction with the results of the methods herein in order to make a diagnosis.

The methods described herein are also useful for assessing the likelihood (or risk) of, or aiding in assessing the likelihood (or risk) of, a subject having or developing dementia or dementia related neurological disorder (e.g., AD, HD or PSP). To practice the methods levels of a plurality of biomarkers are typically determined. These levels are compared to a reference wherein the levels or ratios of the plurality of biomarkers in comparison to the reference levels or ratios is indicative of the likelihood that the subject will develop dementia or dementia related neurological disorder (e.g., AD, HD or PSP).

Other criteria for assessing likelihood that are known in the art (e.g., family history) can also be evaluated in conjunction with the methods described herein in order to make a complete likelihood assessment.

The present methods can also be used for selecting a treatment and/or determining a treatment plan for a subject, based on the occurrence or levels of certain biomarkers relevant to AD, PSP or HD. In some embodiments, using the method disclosed herein, a health care provider (e.g., a physician) identifies a subject as being at risk of having AD, PSP or HD and, based on this identification the health care provider determines an adequate management plan for the subject. In some embodiments, using the method disclosed herein, a health care provider (e.g., a physician) diagnoses a subject as being at risk of having AD, PSP or HD based on the occurrence or levels of certain biomarkers in a clinical sample obtained from the subject, and/or based on a classification of a clinical sample obtained from the subject. By way of this diagnosis the health care provider determines an adequate treatment or treatment plan for the subject. In some embodiments, the methods further include administering the treatment to the subject.

The methods can further comprise selecting, and optionally administering, a treatment for the subject based on the diagnosis (i.e., based on the comparison of the levels of the biomarkers with the reference levels). The treatment can include, for example, administering to the subject an effective amount of at least one anti-dementia compound. Anti-dementia compound are well known in the art and some are disclosed herein. Non-limiting examples include donepezil, memantine, rivastigmine, galanthamine, tacrine, or salts thereof. When a therapeutic agent or other treatment is administered, it is administered in an amount effective to treat dementia or a dementia related neurological disorder or reduce the likelihood (or risk) of future dementia or a dementia related neurological disorder. An effective amount is a dosage of the therapeutic agent sufficient to provide a medically desirable result. The effective amount will vary with the particular condition being treated, the age and physical condition of the subject being treated, the severity of the condition, the duration of the treatment, the nature of the concurrent therapy (if any), the specific route of administration and the like factors within the knowledge and expertise of the health care practitioner. For example, an effective amount can depend upon the degree to which a subject has abnormal levels of certain analytes (e.g., biomarkers as described herein) that are indicative of dementia or a dementia related neurological disorder. It should be understood that the therapeutic agents of the invention are used to treat and/or prevent dementia or a dementia related neurological disorder. Thus, in some cases, they may be used prophylactically in human subjects at risk of developing dementia or a dementia related neurological disorder. Thus, in some cases, an effective amount is that amount which can lower the risk of, slow or perhaps prevent altogether the development of a dementia or a dementia related neurological disorder. It will be recognized when the therapeutic agent is used in acute circumstances, it is used to prevent one or more medically undesirable results that typically flow from such adverse events. Methods for selecting a suitable treatment and an appropriate dose thereof will be apparent to one of ordinary skill in the art.

In some embodiments, the invention relates to identifying subjects who are likely to have successful treatment with a particular drug dose, formulation and/or administration modality. Other embodiments include evaluating the efficacy of a drug using the biomarker profiling methods of the present invention. In some embodiments, the biomarker profiling methods are useful for identifying subjects who are likely to have successful treatment with a particular drug or therapeutic regiment. For example, during a study (e.g., a clinical study) of a drug or treatment, symptomatic subjects who may respond well to the drug or treatment, and others may not. Disparity in treatment efficacy is associated with numerous variables, for example genetic variations among the subjects. In some embodiments, subjects in a population are stratified based on the biomarker profiling methods disclosed herein. In some embodiments, resulting strata are further evaluated based on various epidemiological, and or clinical factors (e.g., response to a specific treatment). In some embodiments, stratum, identified based on a biomarker profile, reflect a subpopulation of subjects that response predictably (e.g., have a predetermined response) to certain treatments. In further embodiments, samples are obtained from subjects who have been subjected to the drug being tested and who have a predetermined response to the treatment. In some cases, a reference can be established from all or a portion of the biomarkers from these samples, for example, to provide a reference metabolic profile. A sample to be tested can then be evaluated (e.g., using a prediction model) against the reference and classified on the basis of whether treatment would be successful or unsuccessful. A company and/or person testing a treatment (e.g., compound, drug, and life-style change) could discern more accurate information regarding the types or subtypes of dementia for which a treatment is most useful. This information also aids a healthcare provider in determining the best treatment plan for a subject.

### Communication to Subject:

The invention further provides for the communication of assay results or diagnoses or both to technicians, physicians or patients, for example. In certain embodiments, computers will be used to communicate assay results or diagnoses or both to interested parties, e.g., physicians and their patients.

In some embodiments of the invention, a diagnosis based on the presence or absence in a test subject of any the biomarkers of Tables 1-4 is communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications.

### Methods of Screening (Test Compounds)

The methods of the present invention have other applications as well. For example, the biomarkers can be used to screen for compounds (e.g., small molecules, inhibitory nucleic acids, antibodies, and peptides) that modulate the expression of the biomarkers in vitro or in vivo, which compounds in turn may be useful in treating or preventing dementia (e.g., AD, HD or PSP) in patients. In another example, the biomarkers can be used to monitor the response to treatments for dementia (e.g., AD, HD or PSP). In yet another example, the biomarkers can be used in heredity studies to determine if the subject is at risk for developing dementia (e.g., AD, HD or PSP).

Thus, included herein are methods for screening test compounds, e.g., polypeptides, polynucleotides, inorganic or organic large or small molecule test compounds, to identify agents useful in the treatment of disorders associated with dementia e.g., AD, HD or PSP.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

Exemplary inhibitory nucleic acids include, but are not limited to, siRNA and antisense nucleic acids.

An “antisense” nucleic acid can include a nucleotide sequence that is complementary to a “sense” nucleic acid encoding a protein, e.g., complementary to the coding strand of a double-stranded cDNA molecule or complementary to a mRNA sequence. The antisense nucleic acid can be complementary to an entire coding strand of a target sequence, or to only a portion thereof. In another embodiment, the antisense nucleic acid molecule is antisense to a “noncoding region” of the coding strand of a nucleotide sequence (e.g., the 5′ and 3′ untranslated regions).

An antisense nucleic acid can be designed such that it is complementary to the entire coding region of a target mRNA, but can also be an oligonucleotide that is antisense to only a portion of the coding or noncoding region of the target mRNA. For example, the antisense oligonucleotide can be complementary to the region surrounding the translation start site of the target mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection). Based upon the sequences disclosed herein, one of skill in the art can easily choose and synthesize any of a number of appropriate antisense molecules for use in accordance with the present invention. For example, a “gene walk” comprising a series of oligonucleotides of 15-30 nucleotides spanning the length of a target nucleic acid can be prepared, followed by testing for inhibition of target gene expression. Optionally, gaps of 5-10 nucleotides can be left between the oligonucleotides to reduce the number of oligonucleotides synthesized and tested. Such methods can also be used to identify siRNAs.

The test compounds can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of small molecule libraries are commercially available.

Libraries screened using the methods of the present invention can comprise a variety of types of test compounds. A given library can comprise a set of structurally related or unrelated test compounds. In some embodiments, the test compounds are peptide or peptidomimetic molecules. In some embodiments, the test compounds are nucleic acids.

In some embodiments, the test compounds and libraries thereof can be obtained by systematically altering the structure of a first test compound, e.g., a first test compound that is structurally similar to a known natural binding partner of the target polypeptide, or a first small molecule identified as capable of binding the target polypeptide, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in one embodiment, a general library of small molecules is screened, e.g., using the methods described herein.

In some embodiments, a test compound is applied to a test sample, e.g., a cell or living tissue or organ, and one or more effects of the test compound is evaluated.

In some embodiments, the test sample is, or is derived from (e.g., a sample taken from) an in vivo model of a disorder as described herein. For example, an animal model, e.g., a rodent such as a rat, can be used.

Methods for evaluating each of these effects are known in the art. For example, ability to modulate expression of a protein can be evaluated at the gene or protein level, e.g., using quantitative PCR or immunoassay methods. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect an effect on gene expression.

A test compound that has been screened by a method described herein and determined to alter the expression level or amount of one or more protein biomarker listed in Tables 1-4, can be considered a candidate compound. A candidate compound that has been screened, e.g., in an in vivo model of a disorder, e.g., an AD mouse model, a HD mouse model or a PSP mouse model, and determined to have a desirable effect on the disorder, e.g., on one or more symptoms of the disorder, can be considered a candidate therapeutic agent. Candidate therapeutic agents, once screened in a clinical setting, are therapeutic agents. Candidate compounds, candidate therapeutic agents, and therapeutic agents can be optionally optimized and/or derivatized, and formulated with physiologically acceptable excipients to form pharmaceutical compositions.

Thus, test compounds identified as “hits” (e.g., test compounds that alter the expression level or amount one or more protein biomarker listed in Tables 1-4) in a first screen can be selected and systematically altered, e.g., using rational design, to optimize binding affinity, avidity, specificity, or other parameter. Such optimization can also be screened for using the methods described herein. Thus, in one embodiment, the invention includes screening a first library of compounds using a method known in the art and/or described herein, identifying one or more hits in that library, subjecting those hits to systematic structural alteration to create a second library of compounds structurally related to the hit, and screening the second library using the methods described herein.

Test compounds identified as hits can be considered candidate therapeutic compounds, useful in treating disorders associated with dementia, as described herein, e.g., AD, HD or PSP. A variety of techniques useful for determining the structures of “hits” can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disorder described herein.

Test compounds identified as candidate therapeutic compounds can be further screened by administration to an animal model of a disorder associated with dementia, as described herein. The animal can be monitored for a change in the disorder, e.g., for an improvement in a parameter of the disorder, e.g., a parameter related to clinical outcome.

Dosage, toxicity, and therapeutic efficacy of the Test compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are typically preferred. While compounds that exhibit toxic side effects may be used, care should be taken to design a delivery system that targets such compounds to the site of affected tissue to minimize potential damage to uninfected cells and, thereby, reduce side effects.

The data obtained from cell culture assays and animal studies can be used in formulating a range of dosages for use in humans. The dosage of such compounds lies preferably within a range of circulating concentrations that include the ED50 with little or no toxicity. The dosage may vary within this range depending upon the dosage form employed and the route of administration utilized. For any compound used in the methods of the inventions described herein, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

### Kits

The invention also provides kits for evaluating biomarkers in a subject. The kits of the invention can take on a variety of forms. Typically, the kits will include reagents suitable for determining levels of a plurality of biomarkers (e.g., those disclosed herein, for example as outlined in Tables 1-4) in a sample. Optionally, the kits may contain one or more control samples or references. Typically, a comparison between the levels of the biomarkers in the subject and levels of the biomarkers in the control samples is indicative of a clinical status (e.g., diagnosis, etc.). Also, the kits, in some cases, will include written information (indicia) providing a reference (e.g., predetermined values), wherein a comparison between the levels of the biomarkers in the subject and the reference (pre-determined values) is indicative of a clinical status. In some cases, the kits comprise software useful for comparing biomarker levels or occurrences with a reference (e.g., a prediction model). Usually the software will be provided in a computer readable format such as a compact disc, but it also may be available for downloading via the internet. However, the kits are not so limited and other variations with will apparent to one of ordinary skill in the art.

### EXAMPLES

The invention is further described in the following examples, which do not limit the scope of the invention described in the claims.

### Example 1

Mass spectrometry was used to compare the concentration of proteins in cerebrospinal fluid (CSF) obtained from 6 control patients (aged 50-65 years), 6 Alzheimer's patients (aged 50-65 years), and 6 Progressive Supranuclear Palsy patients (aged 50-65 years).

A novel platform for the analysis of CSF using quantitative mass spectrometry technique and tandem mass tags (TMT) was used to quantify these CSF proteins in an unbiased manner. To identify proteins that are changed in dementia cases relative to controls, CSF samples were filtered on a 30 kDa membrane to remove interfering peptides, digested with trypsin, isotopically labeled with TMT reagents, then mixed prior to mass spectrometry analysis. Under this regime, the relative abundance of the TMT mass tags corresponds to relative amounts of peptides/proteins in each CSF sample. A total of 655 proteins were compared and quantified at a 1% false discovery rate. Then 2 statistical tests were performed to identify proteins that were significantly different in: a) control vs. Alzheimer's CSF and b) control vs. PSP CSF. Based on these tests, 174 proteins were identified that were differentially regulated in PSP CSF and 67 proteins that were differentially regulated in Alzheimer's CSF with a fold change that is greater that 1.2-fold versus control CSF (Table 1 and Table 2).

TABLE 1
PSP proteins with fold changes above 1.2

| | Gene | Fold Change | |
|---|---|---|---|
| Protein Name | Name | Exp. 1 | Exp. 2 |
| 4F2 cell-surface antigen heavy chain | 4F2 | N/I* | −1.32 |
| Neuroendocrine protein 7B2 | 7B2 | N/I | −1.41 |
| Alpha-1-acid glycoprotein 1 | A1AG1 | 1.44 | 1.23 |
| Alpha-1-antitrypsin | A1AT | 1.51 | N/I |
| Alpha-1B-glycoprotein | A1BG | 1.31 | N/I |
| Alpha-2-macroglobulin | A2MG | N/I | −1.23 |
| Amyloid beta A4 protein | A4 | −1.62 | N/I |
| Alpha-1-antichymotrypsin | AACT | 1.23 | 1.27 |
| Disintegrin and metalloproteinase domain-containing | ADA22 | −1.69 | N/I |
| protein 22 | | | |
| Afamin | AFAM | 1.24 | N/I |
| Peptidyl-glycine alpha-amidating monooxygenase | AMD | −1.48 | −1.32 |
| Amyloid-like protein 1 | APLP1 | −1.30 | N/I |
| Apolipoprotein A-I | APOA1 | 1.25 | N/I |
| Apolipoprotein A-IV | APOA4 | N/I | −1.23 |
| Apolipoprotein E | APOE | −1.42 | N/I |
| N-acetyllactosaminide beta-1,3-N- | B3GN1 | −1.22 | N/I |
| acetylglucosaminyltransferase | | | |
| Biotinidase | BTD | −1.23 | N/I |
| complement C1r subcomponent-like protein | C1RL | N/I | 1.52 |
| Voltage-dependent calcium channel subunit alpha- | CA2D1 | N/I | −1.27 |
| 2/delta-1 | | | |
| Voltage-dependent calcium channel subunit alpha- | CA2D1 | −1.37 | N/I |
| 2/delta-1 | | | |
| Cadherin-13 | CAD13 | N/I | −1.27 |
| Cell adhesion molecule 1 | CADM1 | −1.62 | N/I |
| Cell adhesion molecule 3 | CADM3 | −1.46 | N/I |
| Carbonic anhydrase 1 | CAH1 | N/I | 1.23 |
| Calreticulin | CALR | −1.42 | N/I |
| Soluble calcium-activated nucleotidase 1 | CANT1 | N/I | −1.52 |
| Catalase | CATA | N/I | −1.32 |
| Cathepsin F | CATF | −1.57 | −1.32 |
| Complement factor B | CFAB | 1.32 | N/I |
| complement factor D | CFAD | N/I | −1.27 |
| Neural cell adhesion molecule L1-like protein | CHL1 | −1.59 | −1.27 |
| Chromogranin-A | CMGA | −1.47 | −1.27 |
| Uncharacterized protein C14orf37 | CN037 | −1.50 | N/I |
| Condensin-2 complex subunit D3 | CNDD3 | 1.29 | N/I |
| Beta-Ala-His dipeptidase | CNDP1 | −1.34 | N/I |
| Ciliary neurotrophic factor receptor subunit alpha | CNTFR | −1.37 | N/I |
| Collagen alpha-2(I) chain | CO1A2 | −1.20 | −1.23 |
| Complement C4-A | CO4A | 1.36 | N/I |
| Complement component C6 | CO6 | 1.20 | N/I |
| Collagen alpha-1(VI) chain | CO6A1 | −1.43 | N/I |
| Complement component C8 beta chain | CO8B | 1.22 | N/I |
| Complement component C9 | CO9 | 1.35 | N/I |
| Collectin-12 | COL12 | −1.28 | N/I |
| UPF0764 protein C16orf89 | CP089 | −1.86 | N/I |
| Cartilage acidic protein 1 | CRAC1 | −1.31 | −1.23 |
| Chondroitin sulfate proteoglycan 5 | CSPG5 | −1.59 | N/I |
| Calsyntenin-1 | CSTN1 | −1.59 | N/I |
| Calsyntenin-2 | CSTN2 | N/I | −1.32 |
| Cystatin-C | CYTC | −1.28 | −1.23 |
| Dystroglycan | DAG1 | N/I | −1.27 |
| Dihydropteridine reductase | DHPR | −1.44 | N/I |
| Deleted in autism-related protein 1 | DIA1R | N/I | −1.41 |
| Dickkopf-related protein 3 | DKK3 | −1.21 | N/I |
| Delta and Notch-like epidermal growth factor-related | DNER | −1.32 | N/I |
| receptor | | | |
| Extracellular matrix protein 1 | ECM1 | −1.28 | −1.27 |
| Endonuclease domain-containing 1 protein | ENDD1 | N/I | −1.23 |
| Ephrin type-A receptor 4 | EPHA4 | −1.49 | N/I |
| Coagulation factor XIII A chain | F13A | N/I | −1.57 |
| Coagulation factor IX | FA9 | N/I | −1.27 |
| Protein FAM3C | FAM3C | −1.56 | N/I |
| Protocadherin Fat 2 | FAT2 | N/I | −1.41 |
| Fibulin-7 | FBLN7 | N/I | −1.23 |
| Alpha-2-HS-glycoprotein | FETUA | 1.22 | N/I |
| Complement factor H-related protein 2 | FHR2 | 1.34 | N/I |
| Fibrinogen beta chain | FIBB | N/I | −1.27 |
| Fibronectin | FINC | −1.35 | N/I |
| Tissue alpha-L-fucosidase | FUCO | N/I | −1.23 |
| Plasma alpha-L-fucosidase | FUCO2 | N/I | −1.27 |
| GDNF family receptor alpha-2 | GFRA2 | −1.68 | N/I |
| Transcriptional activator GLI3 | GLI3 | −1.62 | N/I |
| Golgi membrane protein 1 | GOLM1 | −1.77 | −1.41 |
| Glutathione peroxidase 3 | GPX3 | N/I | −1.27 |
| Glutamate receptor 4 | GRIA4 | N/I | −1.23 |
| Hemoglobin subunit beta | HBB | N/I | 1.93 |
| Hemoglobin subunit delta | HBD | N/I | −1.52 |
| Hemopexin | HEMO | 1.31 | N/I |
| Haptoglobin | HPT | 1.98 | 1.27 |
| Ig heavy chain V-III region GAL | HV320 | N/I | −1.23 |
| Insulin-like growth factor-binding protein 7 | IBP7 | 1.22 | 1.23 |
| Ig alpha-1 chain C region | IGHA1 | N/I | −2.30 |
| Ig alpha-2 chain C region | IGHA2 | N/I | −1.68 |
| Ig mu chain C region | IGHM | 1.59 | N/I |
| Immunoglobulin J chain | IGJ | N/I | −1.23 |
| Immunoglobulin lambda-like polypeptide 5 | IGLL5 | N/I | −1.41 |
| Immunoglobulin superfamily member 8 | IGSF8 | −1.30 | N/I |
| Inositol monophosphatase 3 | IMPA3 | N/I | −1.37 |
| Keratin, type I cytoskeletal 14 | K1C14 | −1.45 | N/I |
| Keratin, type I cytoskeletal 16 | K1C16 | −1.36 | N/I |
| Keratin, type I cytoskeletal 17 | K1C17 | −1.78 | N/I |
| Keratin, type I cytoskeletal 9 | K1C9 | 1.35 | N/I |
| Keratin, type II cytoskeletal 1 | K2C1 | 1.40 | N/I |
| Kallistatin | KAIN | N/I | −1.27 |
| Kallikrein-6 | KLK6 | −1.27 | N/I |
| Pyruvate kinase PKM | KPYM | −1.22 | N/I |
| Ig kappa chain V-I region EU | KV106 | N/I | −1.23 |
| Ig kappa chain V-III region POM | KV306 | N/I | −1.41 |
| Ig kappa chain V-III region VG (Fragment) | KV309 | 1.60 | N/I |
| Neural cell adhesion molecule L1 | L1CAM | N/I | −1.27 |
| Laminin subunit alpha-2 | LAMA2 | N/I | −1.37 |
| Serine/threonine-protein kinase LATS2 | LATS2 | −1.88 | N/I |
| Phosphatidylcholine-sterol acyltransferase | LCAT | −1.32 | N/I |
| L-lactate dehydrogenase A chain | LDHA | −1.73 | N/I |
| Lactoylglutathione lyase | LGUL | N/I | −1.41 |
| LINE-1 type transposase domain-containing protein | LITD1 | 1.22 | N/I |
| 1Vwf | | | |
| Latent-transforming growth factor beta-binding | LTBP4 | N/I | −1.23 |
| protein 4 | | | |
| Lumican | LUM | N/I | −1.27 |
| Ig lambda chain V-I region WAH | LV106 | N/I | −1.57 |
| Lymphocyte antigen 6H | LY6H | −1.24 | N/I |
| Lymphatic vessel endothelial hyaluronic acid receptor | LYVE1 | −1.26 | −1.57 |
| 1 | | | |
| Mannosyl-oligosaccharide 1,2-alpha-mannosidase IA | MA1A1 | −1.32 | N/I |
| Multiple epidermal growth factor-like domains | MEGF8 | −1.54 | N/I |
| protein 8 | | | |
| Cell surface glycoprotein MUC18 | MUC18 | −1.45 | N/I |
| Neuroblastoma suppressor of tumorigenicity 1 | NBL1 | −1.32 | N/I |
| Neural cell adhesion molecule 1 | NCAM1 | −1.35 | N/I |
| Neural cell adhesion molecule 2 | NCAM2 | −1.37 | N/I |
| Neurocan core protein | NCAN | −1.46 | N/I |
| Neuroendocrine convertase 1 | NEC1 | N/I | −2.00 |
| Neuronal growth regulator 1 | NEGR1 | −1.64 | N/I |
| Protein kinase C-binding protein NELL2 | NELL2 | −1.50 | N/I |
| Neogenin | NEO1 | −1.88 | N/I |
| Neuroserpin | NEUS | N/I | −1.23 |
| Neurofascin | NFASC | −1.34 | N/I |
| Neuronal pentraxin-1 | NPTX1 | −1.60 | N/I |
| Neuronal pentraxin receptor | NPTXR | −1.50 | −1.32 |
| Neuronal cell adhesion molecule | NRCAM | −1.54 | −1.27 |
| Neuritin | NRN1 | N/I | −1.23 |
| Neuropilin-1 | NRP1 | −1.50 | N/I |
| Neurexin-2-alpha | NRX2A | −1.32 | N/I |
| Neurexin-3-alpha | NRX3A | −1.42 | N/I |
| Oligodendrocyte-myelin glycoprotein | OMGP | −1.44 | N/I |
| Phosphoinositide-3-kinase-interacting protein 1 | P3IP1 | −2.10 | N/I |
| Protocadherin-17 | PCD17 | N/I | −1.27 |
| Protocadherin gamma-C5 | PCDGM | N/I | −1.27 |
| ProSAAS | PCSK1 | −1.34 | N/I |
| Phosphatidylethanolamine-binding protein 4 | PEBP4 | −1.30 | N/I |
| Brevican core protein | PGCB | −1.33 | N/I |
| Plexin-B2 | PLXB2 | N/I | −1.23 |
| Peroxiredoxin-2 | PRDX2 | N/I | −1.32 |
| Proteoglycan 4 | PRG4 | N/I | −1.46 |
| Major prion protein | PRIO | −1.47 | N/I |
| Prostaglandin-H2 D-isomerase | PTGDS | −1.26 | N/I |
| Receptor-type tyrosine-protein phosphatase gamma | PTPRG | −1.59 | N/I |
| Receptor-type tyrosine-protein phosphatase-like N | PTPRN | N/I | −1.52 |
| Receptor-type tyrosine-protein phosphatase zeta | PTPRZ | −1.42 | N/I |
| Glutaminyl-peptide cyclotransferase | QPCT | N/I | −1.37 |
| Sulfhydryl oxidase 2 | QSOX2 | N/I | −1.41 |
| Ribonuclease pancreatic | RNAS1 | −1.26 | N/I |
| Proactivator polypeptide | SAP | −1.33 | N/I |
| Secretogranin-1 | SCG1 | −1.90 | −1.27 |
| Secretogranin-2 | SCG2 | −1.42 | −1.62 |
| Secretogranin-3 | SCG3 | −1.47 | N/I |
| Scrapie-responsive protein 1 | SCRG1 | N/I | −1.27 |
| Seizure 6-like protein | SE6L1 | −1.37 | −1.23 |
| Seizure protein 6 homolog | SEZ6 | N/I | −1.37 |
| Tyrosine-protein phosphatase non-receptor type | SHPS1 | −1.57 | −1.27 |
| substrate 1 | | | |
| SLIT and NTRK-like protein 1 | SLIK1 | −1.39 | N/I |
| Somatostatin | SMS | N/I | −1.52 |
| Superoxide dismutase [Mn], mitochondrial | SODM | −1.65 | N/I |
| Sortilin-related receptor | SORL | N/I | −1.23 |
| SPARC | SPRC | 1.37 | N/I |
| SPARC-like protein 1 | SPRL1 | −1.35 | N/I |
| Sushi domain-containing protein 5 | SUSD5 | −1.27 | N/I |
| Tenascin-X | TENX | −1.56 | N/I |
| Trans-Golgi network integral membrane protein 2 | TGON2 | −1.33 | N/I |
| Thyroxine-binding globulin | THBG | 1.58 | N/I |
| Testican-2 | TICN2 | N/I | −1.57 |
| Tumor necrosis factor receptor superfamily member | TNR21 | N/I | −1.23 |
| 21 | | | |
| Triggering receptor expressed on myeloid cells 2 | TREM2 | N/I | −1.32 |
| V-type proton ATPase subunit S1 | VAS1 | N/I | −1.32 |
| Neurosecretory protein VGF | VGF | −1.67 | −1.41 |
| Vitamin D-binding protein | VTDB | 1.24 | N/I |
| V-set and transmembrane domain-containing protein | VTM2A | −1.35 | N/I |
| 2A | | | |
| V-type proton ATPase subunit S1 | VAS1 | 2.12 | N/I |
| von Willebrand factor | VWF | N/I | −1.93 |
| WAP, kazal, immunoglobulin, kunitz and NTR domain- | WFKN2 | −1.48 | N/I |
| containing protein 2 | | | |

*= Not Investigated

TABLE 2
Alzheimer's proteins with fold changes above 1.2

| | Gene | Fold Change | |
|---|---|---|---|
| Protein Name | Name | Exp. 1 | Exp. 2 |
| Alpha-1-acid glycoprotein 2 | A1AG2 | −1.29 | N/I |
| Amyloid beta A4 protein | A4 | 1.22 | N/I |
| Actin, cytoplasmic 1 | ACTB | 1.29 | N/I |
| Apolipoprotein B-100 | APOB | N/I | −1.27 |
| Complement C1q tumor necrosis factor-related | C1QT3 | 2.25 | N/I |
| protein 3 | | | |
| Voltage-dependent calcium channel subunit | CA2D1 | N/I | −1.27 |
| alpha-2/delta-1 | | | |
| Cell adhesion molecule 4 | CADM4 | 1.25 | N/I |
| Carbonic anhydrase 1 | CAH1 | N/I | −1.37 |
| Carbonic anhydrase 4 | CAH4 | N/I | −1.26 |
| Cathepsin F | CATF | N/I | −1.27 |
| Pro-cathepsin H | CATH | N/I | −1.20 |
| Cathepsin L1 | CATL1 | 1.32 | N/I |
| Carboxypeptidase Q | CBPQ | N/I | −1.30 |
| CD59 glycoprotein | CD59 | 1.24 | N/I |
| Contactin-2 | CNTN2 | 1.21 | N/I |
| Collagen alpha-2(VI) chain | CO6A2 | N/I | −1.53 |
| Macrophage colony-stimulating factor 1 receptor | CSF1R | 1.43 | N/I |
| Cystatin-M | CYTM | 1.45 | N/I |
| N(G),N(G)-dimethylarginine | DDAH1 | 2.12 | N/I |
| dimethylaminohydrolase 1 | | | |
| Desmoplakin | DESP | −1.29 | N/I |
| Gamma-enolase | ENOG | 1.45 | N/I |
| Mammalian ependymin-related protein 1 | EPDR1 | N/I | −1.35 |
| Isoform 20 of Fibroblast growth factor receptor 1 | FGFR1 | N/I | −1.23 |
| Isoform Gamma-A of Fibrinogen gamma chain | FIBG | N/I | −1.20 |
| Glucosidase 2 subunit beta | GLU2B | 1.24 | N/I |
| Guanine deaminase | GUAD | 1.71 | N/I |
| Hemoglobin subunit alpha | HBA | −15.41 | −1.25 |
| Hemoglobin subunit delta | HBD | −20.25 | N/I |
| Plasma protease C1 inhibitor | IC1 | 1.20 | N/I |
| Ig alpha-1 chain C region | IGHA1 | −1.69 | N/I |
| Ig gamma-1 chain C region | IGHG1 | −1.38 | −1.23 |
| Immunoglobulin lambda-like polypeptide 5 | IGLL5 | −1.47 | N/I |
| Inter-alpha-trypsin inhibitor heavy chain H4 | ITIH4 | −1.72 | N/I |
| Keratin, type 1 cytoskeletal 16 | K1C16 | −1.74 | N/I |
| Keratin, type 1 cytoskeletal 17 | K1C17 | −1.96 | N/I |
| Mast/stem cell growth factor receptor Kit | KIT | N/I | −1.23 |
| Ig kappa chain V-I region Roy | KV116 | −1.42 | N/I |
| Ig kappa chain V-III region VG (Fragment) | KV309 | −1.43 | N/I |
| Ig lambda-2 chain C regions | LAC2 | −1.38 | N/I |
| Ig lambda chain V-I region NIG-64 | LV104 | −1.37 | N/I |
| Ig lambda chain V-III region SH | LV301 | −1.74 | N/I |
| Malate dehydrogenase, cytoplasmic | MDHC | 1.24 | N/I |
| Neuronal growth regulator 1 | NEGR1 | N/I | −1.38 |
| Neuroserpin | NEUS | 1.56 | N/I |
| Neuroligin-4, X-linked | NLGNX | 1.26 | N/I |
| Protein NOV homolog | NOV | 1.35 | N/I |
| Neuronal pentraxin receptor | NPTXR | N/I | −1.27 |
| Osteomodulin | OMD | N/I | −1.22 |
| Phosphatidylethanolamine-binding protein 1 | PEBP1 | 1.24 | N/I |
| Decorin | PGS2 | 1.47 | N/I |
| Plexin-B2 | PLXB2 | N/I | −1.20 |
| Peroxiredoxin-2 | PRDX2 | N/I | −1.45 |
| Prolargin | PRELP | N/I | −1.26 |
| Prostaglandin-H2 D-isomerase | PTGDS | N/I | −1.23 |
| Receptor-type tyrosine-protein phosphatase S | PTPRS | 1.20 | N/I |
| Sulfhydryl oxidase 2 | QSOX2 | N/I | −1.30 |
| Retinoic acid receptor responder protein 2 | RARR2 | 1.41 | N/I |
| Renin receptor | RENR | N/I | −1.33 |
| Ribonuclease pancreatic | RNAS1 | 1.25 | N/I |
| Ribonuclease K6 | RNAS6 | N/I | −1.26 |
| Secretogranin-2 | SCG2 | N/I | −1.27 |
| Scrapie-responsive protein 1 | SCRG1 | 1.24 | N/I |
| Tyrosine-protein phosphatase non-receptor type | SHPS1 | 1.22 | N/I |
| substrate 1 | | | |
| Transmembrane protein 132A | T132A | −4.82 | N/I |
| Transcobalamin-2 | TCO2 | N/I | −1.22 |
| Vascular cell adhesion protein 1 | VCAM1 | N/I | −1.22 |
| Neurosecretory protein VGF | VGF | N/I | −1.32 |

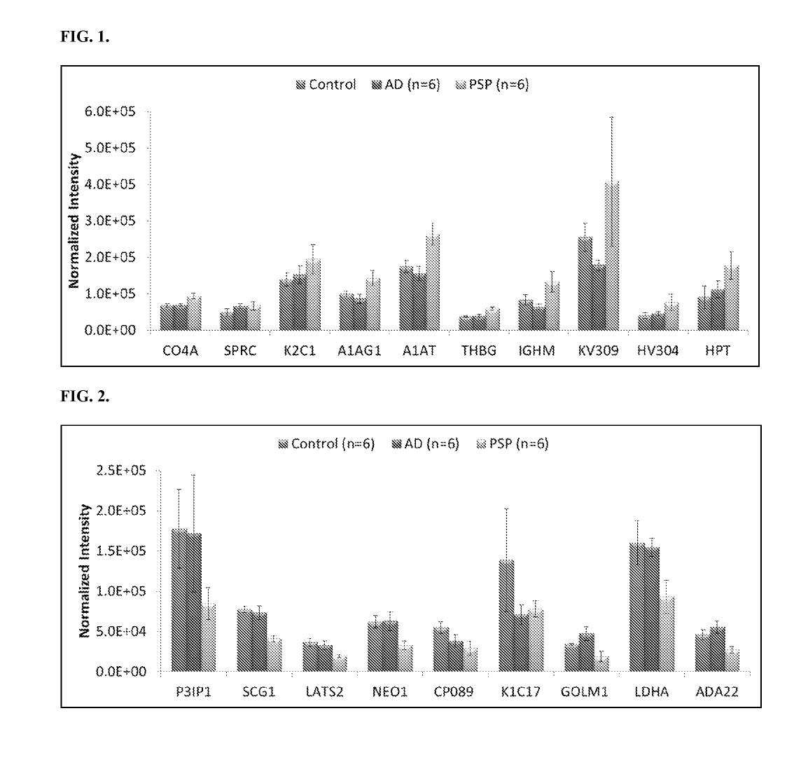

Of these differentially regulated proteins, 110 were uniquely regulated by 1.2 fold in PSP CSF and 39 were uniquely regulated by 1.2 fold in Alzheimer's CSF. When the fold change threshold is increased to 1.5, 49 proteins were found to be differentially regulated in PSP (Table 3). Specifically, when the fold change threshold is increased to 1.5, 6 proteins were up-regulated (A1AT, THBG, IGHM, KV309, HBB and HPT)(FIG. 1) and 43 proteins were down-regulated (A4, ADA22, C1RL, CADM1, CANT1, CATF, CHL1, CN037, CP089, CSPG5, CSTN1, F13A, FAM3C, GFRA2, GLI3, GOLM1, HBD, IGHA1, IGHA2, K1C17, LATS2, LDHA, LYVE1, MEGF8, NEC1, NEGR1, NELL2, NEO1, NPTX1, NPTXR, NRCAM, NRP1, P3IP1, PTPRG, PTPRN, SCG1, SCG2, SHPS1, SMS, SODM, TENX, TICN2, VGF, and VWF)(FIG. 2).

TABLE 3
PSP proteins with fold changes above 1.5

| | Gene | Fold Change | |
|---|---|---|---|
| Protein Name | Name | Exp. 1 | Exp. 2 |
| Alpha-1-antitrypsin | A1AT | 1.51 | N/I |
| Amyloid beta A4 protein | A4 | −1.62 | N/I |
| Disintegrin and metalloproteinase domain- | ADA22 | −1.69 | N/I |
| containing protein 22 | | | |
| complement C1r subcomponent-like protein | C1RL | N/I | 1.52 |
| Cell adhesion molecule 1 | CADM1 | −1.62 | N/I |
| Soluble calcium-activated nucleotidase 1 | CANT1 | N/I | −1.52 |
| Cathepsin F | CATF | −1.57 | −1.32 |
| Neural cell adhesion molecule L1-like protein | CHL1 | −1.59 | −1.27 |
| Uncharacterized protein C14orf37 | CN037 | −1.50 | N/I |
| UPF0764 protein C16orf89 | CP089 | −1.86 | N/I |
| Chondroitin sulfate proteoglycan 5 | CSPG5 | −1.59 | N/I |
| Calsyntenin-1 | CSTN1 | −1.59 | N/I |
| Coagulation factor XIII A chain | F13A | N/I | −1.57 |
| Protein FAM3C | FAM3C | −1.56 | N/I |
| GDNF family receptor alpha-2 | GFRA2 | −1.68 | N/I |
| Transcriptional activator GLI3 | GLI3 | −1.62 | N/I |
| Golgi membrane protein 1 | GOLM1 | −1.77 | −1.41 |
| Hemoglobin subunit beta | HBB | N/I | 1.93 |
| Hemoglobin subunit delta | HBD | N/I | −1.52 |
| Haptoglobin | HPT | 1.98 | 1.27 |
| Ig alpha-1 chain C region | IGHA1 | N/I | −2.30 |
| Ig alpha-2 chain C region | IGHA2 | N/I | −1.68 |
| Ig mu chain C region | IGHM | 1.59 | N/I |
| Keratin, type I cytoskeletal 17 | K1C17 | −1.78 | N/I |
| Ig kappa chain V-III region VG (Fragment) | KV309 | 1.60 | N/I |
| Serine/threonine-protein kinase LATS2 | LATS2 | −1.88 | N/I |
| L-lactate dehydrogenase A chain | LDHA | −1.73 | N/I |
| Lymphatic vessel endothelial hyaluronic acid | LYVE1 | −1.26 | −1.57 |
| receptor 1 | | | |
| Multiple epidermal growth factor-like domains | MEGF8 | −1.54 | N/I |
| protein 8 | | | |
| Neuroendocrine convertase 1 | NEC1 | N/I | −2.00 |
| Neuronal growth regulator 1 | NEGR1 | −1.64 | N/I |
| Protein kinase C-binding protein NELL2 | NELL2 | −1.50 | N/I |
| Neogenin | NEO1 | −1.88 | N/I |
| Neuronal pentraxin-1 | NPTX1 | −1.60 | N/I |
| Neuronal pentraxin receptor | NPTXR | −1.50 | −1.32 |
| Neuronal cell adhesion molecule | NRCAM | −1.54 | −1.27 |
| Neuropilin-1 | NRP1 | −1.50 | N/I |
| Phosphoinositide-3-kinase-interacting protein 1 | P3IP1 | −2.10 | N/I |
| Receptor-type tyrosine-protein phosphatase | PTPRG | −1.59 | N/I |
| gamma | | | |
| Receptor-type tyrosine-protein phosphatase-like N | PTPRN | N/I | −1.52 |
| Secretogranin-1 | SCG1 | −1.90 | −1.27 |
| Secretogranin-2 | SCG2 | −1.42 | −1.62 |
| Tyrosine-protein phosphatase non-receptor type | SHPS1 | −1.57 | −1.27 |
| substrate 1 | | | |
| Somatostatin | SMS | N/I | −1.52 |
| Superoxide dismutase [Mn], mitochondrial | SODM | −1.65 | N/I |
| Thyroxine-binding globulin | THBG | 1.58 | N/I |
| Tenascin-X | TENX | −1.56 | N/I |
| Testican-2 | TICN2 | N/I | −1.57 |
| Neurosecretory protein VGF | VGF | −1.67 | −1.41 |
| von Willebrand factor | VWF | N/I | −1.93 |

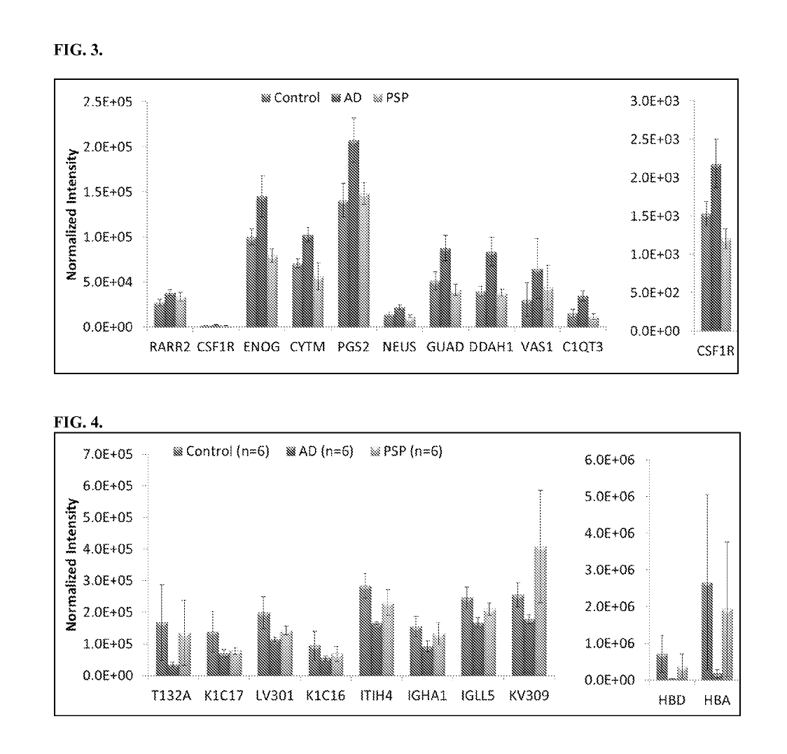

When the fold change threshold is increased to 1.5, 13 proteins were uniquely regulated in AD (Table 4). Of the 13 proteins found to be differentially regulated in AD (>1.5 FC), 5 proteins were up-regulated (NEUS, GUAD, DDAH1, VAS1, and C1QT3)(FIG. 3) and 11 proteins were down-regulated (C06A2, IGHA1, HBD, HBA, T132A, K1C17, LV301, K1C16, ITIH4, IGLL5, and KV309)(FIG. 4). Notably, hemoglobin subunit delta (HBD) and hemoglobin subunit alpha (HBA) were down-regulated with a fold change of greater than 15.

TABLE 4
Alzheimer's proteins with fold changes above 1.5

| | |
|---|---|
| Gene | Fold Change |

| Protein Name | Name | Exp. 1 | Exp. 2 |
|---|---|---|---|
| Complement C1q tumor necrosis | C1QT3 | 2.25 | N/I |
| factor-related protein 3 | | | |
| Collagen alpha-2(VI) chain | CO6A2 | N/I | −1.53 |
| N(G),N(G)-dimethylarginine | DDAH1 | 2.12 | N/I |
| dimethylaminohydrolase 1 | | | |
| Guanine deaminase | GUAD | 1.71 | N/I |
| Hemoglobin subunit alpha | HBA | −15.41 | −1.25 |
| Hemoglobin subunit delta | HBD | −20.25 | N/I |
| Ig alpha-1 chain C region | IGHA1 | −1.69 | N/I |
| Inter-alpha-trypsin inhibitor | ITIH4 | −1.72 | N/I |
| heavy chain H4 | | | |
| Keratin, type 1 cytoskeletal 16 | K1C16 | −1.74 | N/I |
| Keratin, type 1 cytoskeletal 17 | K1C17 | −1.96 | N/I |
| Neuroserpin | NEUS | 1.56 | N/I |
| Transmembrane protein 132A | T132A | −4.82 | N/I |
| Ig lambda chain V-III region SH | LV301 | −1.74 | N/I |

Each biomarker listed in Tables 1 and 3 are differentially present in PSP disease, and, therefore, each is individually useful in aiding in the determination of PSP disease status.

Each biomarker listed in Tables 2 and 4 are differentially present in Alzheimer's disease, and, therefore, each is individually useful in aiding in the determination of Alzheimer's disease status.

Of these proteins, only a single protein, keratin, type 1 cytoskeletal 17, was found to be differentially regulated in both datasets. Haptoglobin, IgG's (kappa and heavy chain V-III), Phosphoinositide-3-kinase-interacting protein, Secretogranin 1, and serine/threonine-protein kinase showed the most significant unique regulation in PSP. Complement C1q necrosis factor, V-type proton ATPase subunit S1, N(G), N(G)-dimethylarginine dimethylaminohydrolase 1, hemoglobin subunits delta and alpha, and transmembrane protein 132A showed the most significant and unique regulation in AD.

The inventors further profiled these significantly different proteins to identify proteins that showed opposing and significant levels of regulation in AD vs. PSP CSF. A comparison of this list revealed that Amyloid beta A4 (A4), Ig kappa chain V-III region VG (fragment) (KV309), Secretogranin-3, Alpha-1-antitrypsin (SCG3), Calsyntenin-1 (CSTN1), Cell adhesion molecule 1 (CADM1), and Tyrosine-protein phosphatase non-receptor type substrate 1 (SHPS1) showed the highest degree of opposing regulation in AD vs. PSP CSF. (FIG. 5)

Several of these proteins are involved in the immune response and complement pathway (see Table 6). All proteins were up-regulated compared to controls.

TABLE 6

| Protein | FC in PSP | FC in AD |
|---|---|---|
| Factor H (related protein 2) | 1.34 | n/a |
| Factor B | 1.32 | n/a |
| CD59 | n/a | 1.24 |
| C4a | 1.36 | n/a |
| C1q tumor necrosis factor | n/a | 2.25 (largest |
| | | observed FC) |
| C6 | 1.20 | n/a |
| C8 | 1.22 | n/a |
| C9 | 1.35 | n/a |

For instance, complement C1q tumor necrosis factor-related protein 3, complement decay accelerating factor, and seizure 6 like protein (i.e., predicted similar to complement factor H-related protein C) are involved in complement activation and were found to be significantly up- or down-regulated in AD CSF. Interestingly, a number of proteins that are involved in immune systems processes were also found to be differentially regulated in AD CSF. Specifically, IgL 2 light chain variable region, cathepsin 1, MHC class I antigen, zinc-alpha-2-glycoprotein, contactins 1 and 2, CD59 glycoprotein, ganglioside GM2 activator, collagen alpha-1(VI) chain, and alpha-1-acid glycoprotein 2 were found to be differentially regulated in AD CSF.

Interestingly, a larger number of complement proteins were found to be differentially regulated in PSP CSF. Specifically, complement component C8 alpha chain, C8 beta chain, component C6, C3a anaphylatoxin, factor B and factor H related protein 2 were found to be differentially regulated with a fold change of 1.2-fold or greater in AD CSF compared to control CSF. Seizure 6-like protein and haptoglobin, proteins that are involved in complement activation, were also found to be differentially regulated in PSP CSF. Additionally, 30 non-complement inflammatory proteins were found to be differentially regulated in PSP CSF compared to control CSF. The largest fold-change for these inflammatory proteins were observed on neogenin, superoxide dismutase, neuronal pentraxin-1, neural cell adhesion molecule L1, cathepsin F, multiple epidermal growth factor like-domains, and neuropilin.

### Example 2

Early synapse loss and dysfunction are becoming increasingly recognized as a hallmark of Alzheimer's disease (AD); however, the factors that trigger synapse loss in the aged and diseased brain remain elusive. There is extensive evidence that soluble Aβ oligomers act as prime synaptotoxic agents in AD; however, what mediates the physical loss of synapses in the AD brain remains unknown. Another major hallmark of AD is the marked increase in neuroinflammatory pathways, which includes the components of the complement cascade and reactive gliosis.

Complement activation is a major inflammatory process whose primary functions are to assist in removing micro-organisms and cellular debris and processing of immune complexes. It may be activated by three pathways: first, via the “classical” activation route through activation of the C1q complex by Ig/antigen immune complexes or non-immune molecules on the surface of dead cells, debris an pathogens, resulting in a downstream cascade ultimately resulting in phagocytosis or cell lysis via membrane-attack complex (FIG. 11); second, via the immune-complex-independent alternative activation pathway leading to deposition of C3 fragments on target cells; and third, via the lectin route by binding mannose-binding lectin to pathogen-associated molecular patterns (PAMPs).

The inventors had previously identified the unexpected role of glia and the complement system in mediating synapse loss during development and disease (glaucoma) (Stevens et al., Cell 2007). However, the role of complement components in synapse loss and their use in early detection of disease has not been evaluated. Thus, the inventors investigated whether early upregulation of complement occurred in vulnerable brain regions and whether complement targeted synapse in mouse models of neurodegenearative disease (e.g., AD brain or Huntington's disease brain).

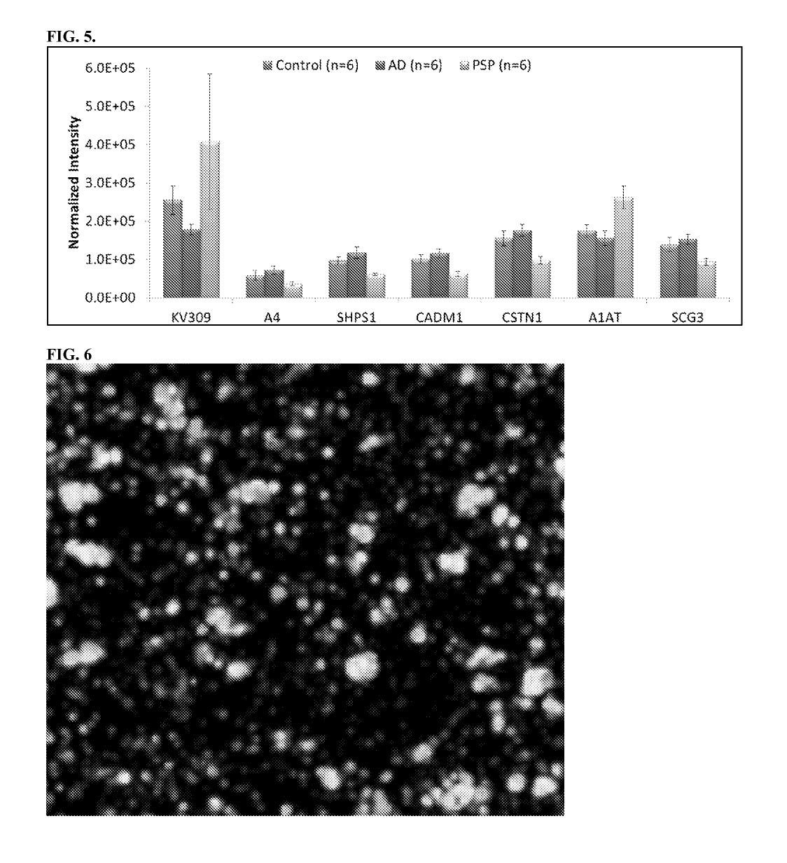

The inventors found that there is an early complement (C1q, C3) upregulation, classical cascade activation, and deposition of complement proteins onto synapses in regions vulnerable to Aβ deposition and synapse loss, the hippocampus and dentate, in a two mouse models of Alzheimer's disease (J20 hAPP TG (described below) and APP PS1 (data not shown)). Specifically, the inventors found that C1q (green) localizes with many pre- and postsynaptic proteins synapsin (white) and PSD95 (red) on a frontal cortex of postmortem AD brain section. (FIG. 6). For FIG. 6, postmortem AD brain tissue (frontal cortex) was briefly fixed in 4% PFA and embedded in LR White resin. Ribbons of 30 serial 70 nm thick sections were mounted on subbed glass coverslips and immunostained with anti-C1q, anti-Synapsin and anti-PSD95. Serial sections were imaged using a Zeiss Imager Z1 microscope, 63× objective and subsequent volumes were aligned using ImageJ (NIH) with the multistackreg plugin (Brad Busse).

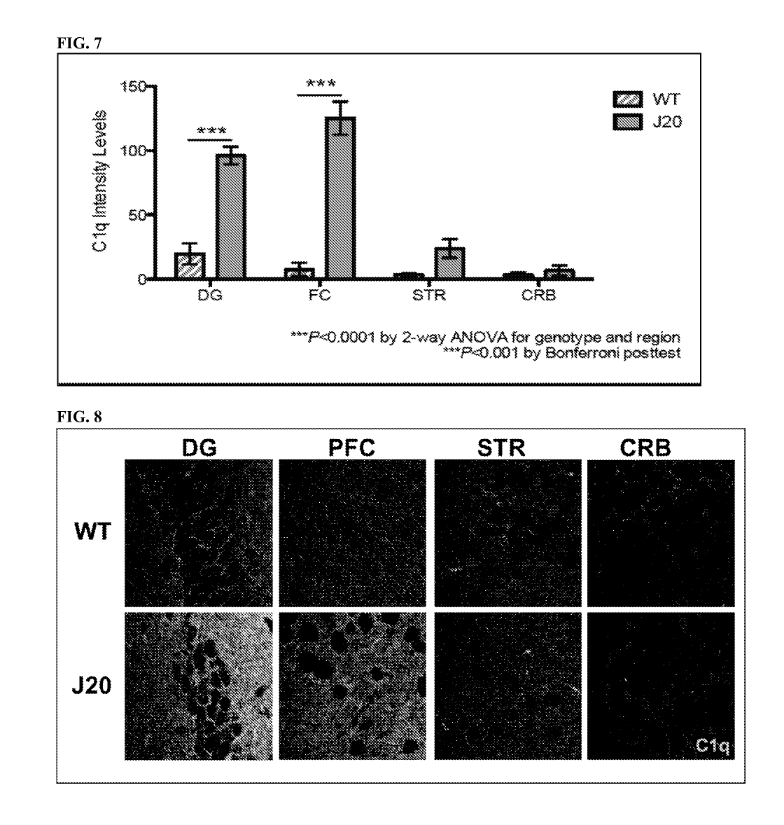

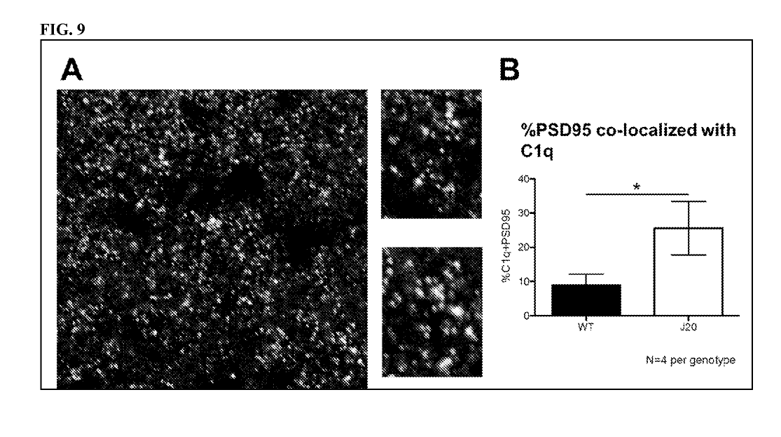

The inventors also found that C1q is specifically upregulated in areas vulnerable to Aβ Deposition in Young (P30) Pre-plaque J20 APP Tg Mice. (FIGS. 7-8). These data demonstrate that C1q is specifically upregulated in areas vulnerable to Aβ (e.g., hippocampus and prefrontal cortex) in young (P30) pre-plaque J20 APP tg mice.

To examine whether C1q is localized to PSD95 in hippocampus of young (P30) pre-plaque J20 APP tg mice, P30 mice were transcardially perfused with PBS followed by 4% PFA, then brains were dissected and postfixed in 4% PFA for 2 h at 4° C. then transferred to 30% sucrose for 24 h. 14 μm cyrostat sections were prepared, then blocked and permeabilized at room temperature for 1 h using BSA and Triton-X 100 followed by anti-C1q and anti-PSD95 antibodies overnight at 4° C. Sections were incubated with an Alexa-fluorophore-conjugated secondary antibody and mounted on slides with vectashield containing DAPI (Vector labs). Images were acquired using a Zeiss LSM 700 Laser Scanning Confocal and Zen 2009 image acquisition software (Carl Zeiss) and analyzed using ImageJ (NIH). The results show that C1q does in fact localize to PSD95 in hippocampus of young (P30) pre-plaque J20 APP tg mice.

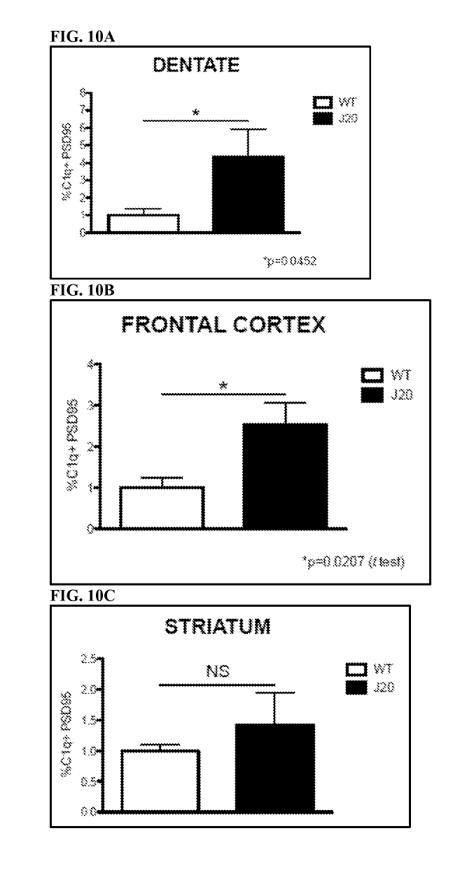

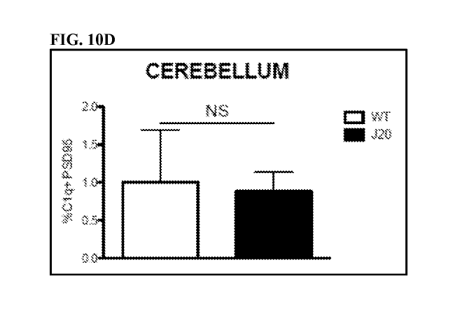

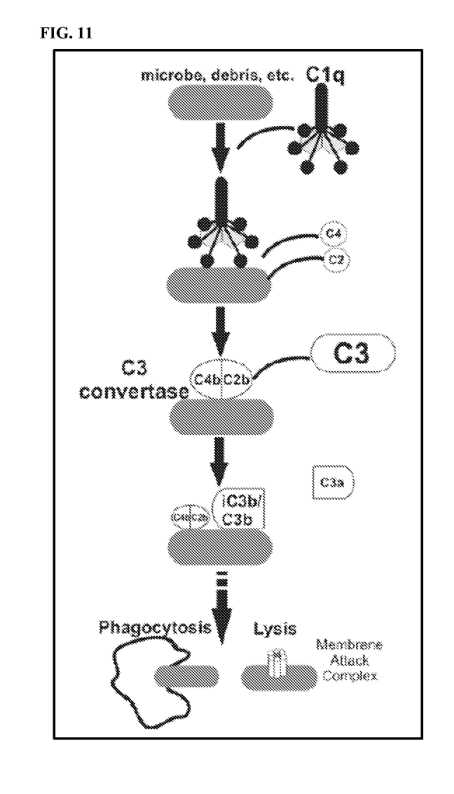

The inventors also examined whether deposition of C1q onto PSD95 was localized to regions areas vulnerable to Aβ Deposition. P30 mice were transcardially perfused with PBS followed by 4% PFA, then brains were dissected and postfixed in 4% PFA for 2 h at 4° C. then transferred to 30% sucrose for 24 h. 14 μm cyrostat sections were prepared, then blocked and permeabilized at room temperature for 1 h using BSA and Triton-X 100 followed by anti-C1q and anti-PSD95 antibodies overnight at 4° C. Sections were incubated with an Alexa-fluorophore-conjugated secondary antibody and mounted on slides with vectashield containing DAPI (Vector labs). Images were acquired using a Zeiss LSM 700 Laser Scanning Confocal and Zen 2009 image acquisition software (Carl Zeiss) and analyzed using ImageJ (NIH). The results demonstrate region-specific deposition of C1q onto PSD95 in the brains of young (P30) pre-plaque J20 mice. (FIGS. 10a-d) Specifically, these data show co-localization of C1q with PSD-95 is significantly higher in the dentate (A) and frontal cortex (B), but not in the striatum (C) or cerebellum (D), of J20 mice vs. littermate WT controls. In addition, the inventors observe an early up-regulation and synaptic deposition of complement C3 (data not shown) indicating early activation of complement cascade in vulnerable brain regions (data not shown).

These findings identify complement-related pathways can serve as early biomarkers that predict disease progression and possibly the onset of cognitive decline and synaptic loss and/or dysfunction in patients with AD, PSP, HD and other neurodegenerative diseases or conditions.

### Example 3

Studies of HD postmortem brains and mouse models have revealed profound loss of synapses in vulnerable cortical and striatal brain regions, however the underlying mechanisms remain elusive. The inventors sought to examiner whether the complement system contributes to HD pathogenesis through early complement and microglia-mediated synaptic loss due to aberrant neural activity in the cortico-striatal pathway.

To study how (full-length human mutant huntingtin) fl-mHTT may elicit selective pathogenesis in HD, a conditional BAC transgenic mouse model of HD (BACHD) that expresses fl-mHTT with 97 Q was developed. These mice exhibit progressive motor deficits (e.g. rotarod, open field) and psychiatric-like deficits (e.g. enhanced anxiety in a light-dark box test and enhanced depression-like behavior in a forced swimming test), which manifest between 2 m and 12 m of age. Importantly, at 12 m of age, BACHD mice exhibit HD-like selective cortical and striatal atrophy (without cerebellar atrophy) and predominantly neuropil mHTT aggregates. BACHD is a widely used fl-mHTT-expressing HD mouse model that partially recapitulates the disease phenotypes and is suitable to study the pathogenesis and treatment of HD.

BACHD mice exhibit early upregulation of complement factor C1q and C3 in the cortico-striatal pathway.

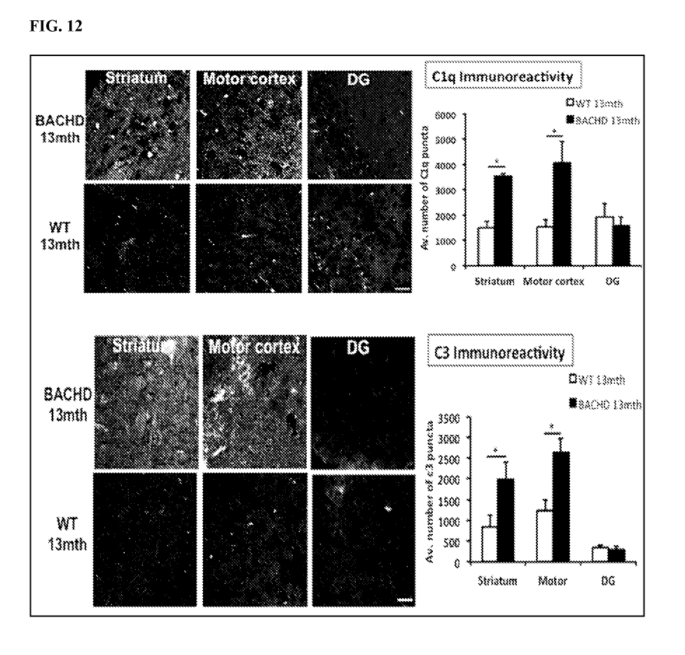

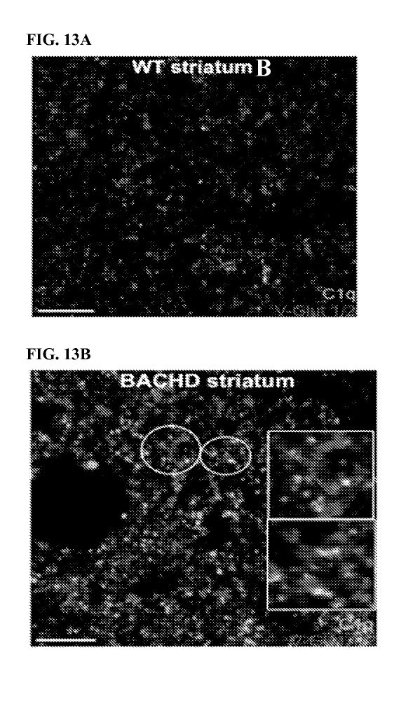

The inventors obtained substantial and converging novel data to show that complement activation and synaptic deposition, as well as microglia-mediated engulfment of synaptic elements, occurs in HD mice. Immunohistochemistry (IHC) studies reveal a significant increase in the total levels of C1q and C3 protein in the motor cortex and striatum of 13 m BACHD mice compared to WT littermate controls (FIG. 12). Interestingly dentate gyrus (DG) showed little differences in levels between the two genotypes (FIG. 12). Consistent with such findings, Fluorescent In Situ Hybridization (FISH) experiments reveal that C1q mRNA is upregulated in Ctip2+ MSNs in the striatum of 7 m BACHD mice (data not shown), suggesting a local upregulation of the initiating protein of the classical complement cascade in the HD-vulnerable neuronal cells.

Complement Deposition at Synapses in BACHD Mice.

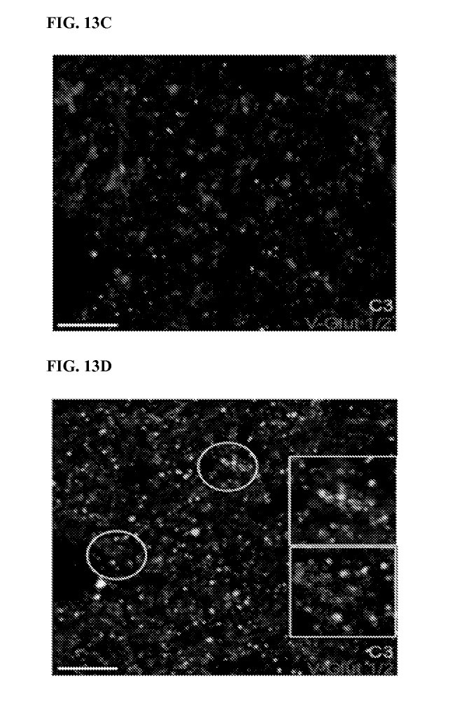

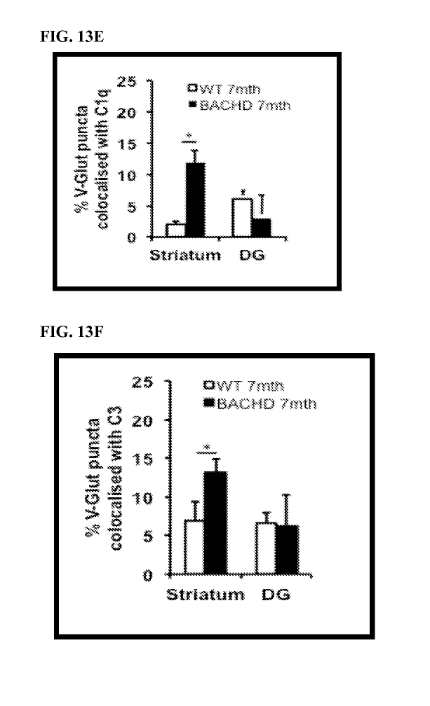

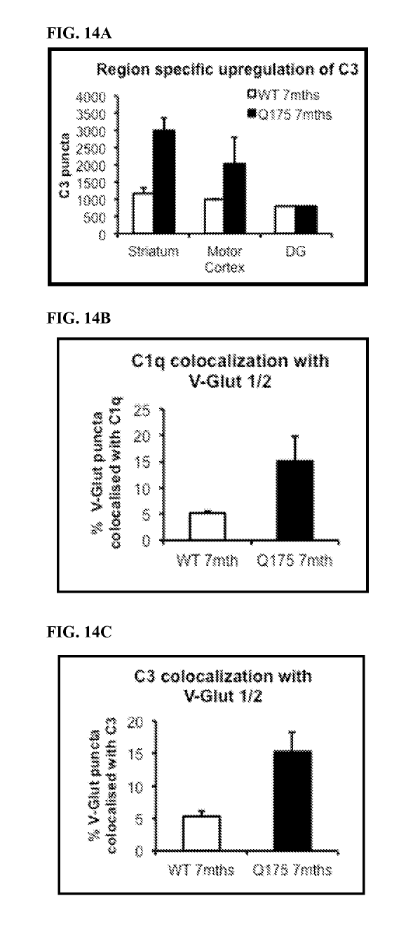

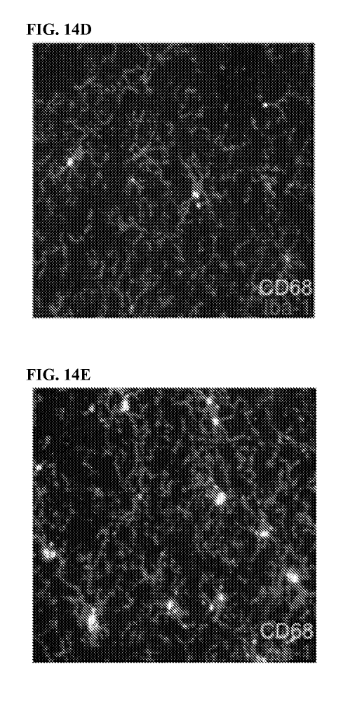

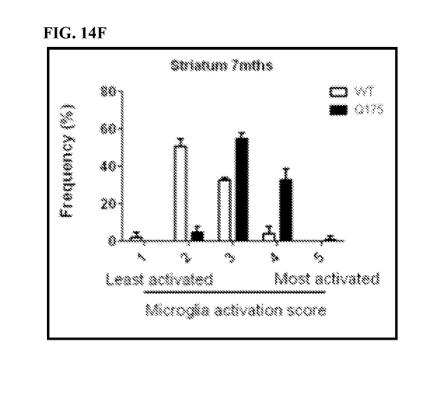

To address whether complement proteins are localized to vulnerable synapses, the inventors performed co-staining with markers of C1q/C3 paired with excitatory synaptic antibodies recognizing both V-Glut1 and V-Glut2 (“V-Glut1/2” antibody) in two HD mouse models (BACHD and zQ175). The results show that in 7 m BACHD mice approximately 15% of V-Glut1/2 puncta in the striatum localized with C3 (FIGS. 13D, F). In contrast little colocalization of C3 and V-Glut1/2 was observed in WT striatum or the DG of either genotype (FIGS. 13C, F). As with C3, a greater percentage of C1q colocalized with synaptic markers in the striatum of 7 m BACHD compared to WT littermate controls (FIGS. 13A, B, E). The results also show similar upregulation and localization in HD-vulnerable brain regions of zQ175 knock-in mice (FIGS. 14A-C), suggesting that complement/microglia activation may play an early role in multiple fl-mHTT-expressing HD mouse models.

The identified markers provide a significant improvement over currently described methods because this is first such investigation that quantifies hundreds of CSF proteins that spans several forms of dementia. Furthermore, the removal of peptide interference prior to TMT-labeling by filter aided sample preparation (FASP), as was done for this study, represents a novel way of quantifying CSF proteins by mass spectrometry that increased the confidence of the proposed biomarker candidates. Several proteins that were uniquely regulated in PSP and AD CSF were identified, which lays the groundwork for the validation of these biomarker candidates using a larger cohort of patients.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for determining the risk of developing dementia in a subject, the method comprising:
obtaining a sample from the subject;
determining a level of one or more biomarkers in said sample;
comparing the levels of the one or more biomarkers with reference levels of the same biomarkers to identify an increase or decrease in a level of said one or more biomarkers is said sample; and
identifying a subject who has an increase or decrease in the level of said one or more biomarkers is said sample as having an increased risk of developing dementia,
wherein the one or more biomarkers are selected from the group of biomarkers listed in Tables 1-4.

2. The method of claim 1, wherein the dementia is Alzheimer's Disease (AD) or Progressive Supernuclear Palsy (PSP).

3. The method of claim 2, comprising determining the levels of one or more of Neuroserpin (NEUS), Guanine deaminase (GUAD), N(G), N(G)-dimethylarginine dimethylaminohydrolase 1 (DDAH1), V-type proton ATPase subunit 1 (VAS1), Complement C1q tumor necrosis factor-related protein 3 (C1QT3), hemoglobin subunit delta (HBD), hemoglobin subunit alpha (HBA), Transmembrane protein 132A (T132A), Keratin, type I cytoskeletal 17 (K1C17), Ig lambda chain V-III region SH (LV301), Keratin, type I cytoskeletal 16 (K1C16), Inter-alpha-trypsin inhibitor heavy chain H4 (ITIH4), Immunoglobulin lambda-like polypeptide 5 (IGLL5), Ig kappa chain V-III region VG (Fragment) (KV309), Collagen alpha-2(VI) chain (C06A2), Ig alpha-1 chain C region (IGHA1), and Ig alpha-1 chain C region (IGHA1).

4. The method of claim 3, comprising identifying a subject who has an increased level of one or more of NEUS, GUAD, DDAH1, VAS1, or Complement C1q tumor necrosis factor-related protein 3 (C1QT3) as having an increased risk of developing Alzheimer's Disease (AD).

5. The method of claim 3, comprising identifying a subject who has a decreased level of one or more of HBD, HBA, T132A, K1C17, LV301, K1C16, ITIH4, IGLL5, KV309, CO6A2, IGHA1, or IGLL5 as having an increased risk of developing Alzheimer's Disease (AD).

6. The method of claim 2, comprising determining the levels of one or more of Phosphoinositide-3-kinase-interacting protein 1 (P3IP1), Secretogranin-1 (SCG1), Serine/threonine-protein kinase LATS2 (LATS2), Neogenin (NEO1), UPF0764 protein C16orf89 (CP089), Keratin, type I cytoskeletal 17 (K1C17), Golgi membrane protein 1 (GOLM1), L-lactate dehydrogenase A chain (LDHA), Disintegrin and metalloproteinase domain-containing protein 22 (ADA22), GDNF family receptor alpha-2 (GFRA2), Neurosecretory protein VGF (VGF), Superoxide dismutase [Mn], mitochondrial (SODM), Neuronal growth regulator 1 (NEGR1), Amyloid beta A4 protein (A4), Cell adhesion molecule 1 (CADM1), Transcriptional activator GLI3 (GLI3), Neuronal pentraxin-1 (NPTX1), Neural cell adhesion molecule L1-like protein (CHL1), Chondroitin sulfate proteoglycan 5 (CSPG5), Receptor-type tyrosine-protein phosphatase gamma (PTPRG), Calsyntenin-1 (CSTN1), Tyrosine-protein phosphatase non-receptor type substrate 1 (SHPS1), Cathepsin F (CATF), Tenascin-X (TENX), Protein FAM3C (FAM3C), Multiple epidermal growth factor-like domains protein 8 (MEGF8), Neuronal cell adhesion molecule (NRCAM), Neuronal pentraxin receptor (NPTXR), Neuropilin-1 (NRP1), Uncharacterized protein C14orf37 (CN037), Protein kinase C-binding protein NELL2 (NELL2), Alpha-1-antitrypsin (AlAT), Thyroxine-binding globulin (THBG), Ig mu chain C region (IGHM), Ig kappa chain V-III region VG (Fragment) (KV309), Collagen alpha-2(VI) chain (CO6A2), Ig alpha-1 chain C region (IGHA1), hemoglobin subunit beta (HBB), Ig heavy chain V-III region TIL (HV304), and Haptoglobin (HPT).

7. The method of claim 6, comprising identifying a subject who has a decreased level of one or more of A4, ADA22, C1RL, CADM1, CANT1, CATF, CHL1, CN037, CP089, CSPG5, CSTN1, F13A, FAM3C, GFRA2, GLI3, GOLM1, HBD, IGHA1, IGHA2, K1C17, LATS2, LDHA, LYVE1, MEGF8, NEC1, NEGR1, NELL2, NEO1, NPTX1, NPTXR, NRCAM, NRP1, P3IP1, PTPRG, PTPRN, SCG1, SCG2, SHPS1, SMS, SODM, TENX, TICN2, VGF, VWF as having an increased risk of developing PSP.

8. The method of claim 6, comprising identifying a subject who has an increased level of one or more of A1AT, THBG, IGHM, KV309, HBB or HPT as having an increased risk of developing PSP.

9. The method of claim 1, further comprising selecting a treatment for the subject based on the comparison of the levels of the biomarkers with the reference levels.

10. The method of claim 9, further comprising administering the selected treatment to the subject.

11. The method of claim 1, further comprising administering to the subject an effective amount of at least one anti-dementia compound.

12. The method of claim 11, wherein the anti-dementia compound is donepezil, memantine, rivastigmine, galanthamine, tacrine, or salts thereof.

13. The method of claim 1, wherein the sample is a biological sample.

14. The method of claim 13, wherein the biological sample is a body fluid, cerebrospinal fluid (CSF), blood, whole blood, plasma, serum, mucus secretions or saliva.

15. The method of claim 13, wherein the biological sample is a cerebrospinal fluid sample (CSF).

16. The method of claim 1, wherein the level of biomarkers are determined using a process selected from the group consisting of mass spectrometry, immunoblotting, ELISA assays, or protein microarrays.

17. The method of claim 1, wherein the reference level of the one or more biomarkers is determined from a sample obtained from a non-demented control subject.

18. The method of claim 1, comprising determining the levels of one or more of complement factors selected from the group consisting of Factor H (related protein 2), C3a anaphylatoxin, C8 alpha chain, C8 beta chain, Factor B, CD59, C4a, C1q tumor necrosis factor, C6, C8 and C9.

19. The method of claim 18, comprising identifying a subject who has an increased level of one or more of Factor H (related protein 2), C8 alpha chain, C8 beta chain, C3a anaphylatoxin Factor B, C4a, C6, C8 or C9 as having an increased risk of developing PSP.

20. The method of claim 18, comprising identifying a subject who has an increased level of one or more of CD59 or C1q tumor necrosis factor as having an increased risk of developing Alzheimer's Disease (AD).
